(19) United States
(12) Patent Application Publication
(10) Pub. No.: US 2022/0396840 A1
(43) Pub. Date: Dec. 15, 2022

(54) IRON-SCORE AND IN VITRO METHOD FOR IDENTIFYING MANTLE CELL LYMPHOMA (MCL) SUBJECTS AND THERAPEUTIC USES AND METHODS

(71) Applicants: CENTRE NATIONAL DE LA RECHERCHE SCIENTIFIQUE (CNRS), Paris (FR); CENTRE HOSPITALIER UNIVERSITAIRE DE MONTPELLIER, Montpellier (FR); INSTITUT NATIONAL DE LA SANTÉ ET DE LA RECHERCHE MÉDICALE (INSERM), Paris (FR); UNIVERSITE DE MONTPELLIER, Montpellier (FR); INSTITUT CURIE, Paris (FR)

(72) Inventors: Jérôme MOREAUX, MONTPELLIER (FR); Raphaël RODRIGUEZ, PARIS (FR); Julie DEVIN, MONTPELLIER (FR); Caroline BRET, MONTPELLIER (FR); Tatiana CANEQUE COBO, PARIS (FR)

(73) Assignees: CENTRE NATIONAL DE LA RECHERCHE SCIENTIFIQUE (CNRS), Paris (FR); CENTRE HOSPITALIER UNIVERSITAIRE DE MONTPELLIER, Montpellier (FR); INSTITUT NATIONAL DE LA SANTÉ ET DE LA RECHERCHE MÉDICALE (INSERM), Paris (FR); UNIVERSITE DE MONTPELLIER, Montpellier (FR); INSTITUT CURIE, Paris (FR)

(21) Appl. No.: 17/774,810

(22) PCT Filed: Nov. 6, 2020
(86) PCT No.: PCT/EP2020/081352

§ 371 Date: May 5, 2022

(30) Foreign Application Priority Data
Nov. 6, 2019 (EP) 19306436.7

(51) Int. Cl. C12Q 1/6886

(52) U.S. Cl. CPC C12Q 1/6886; C12Q 2600/158

(57) ABSTRACT

The invention relates to the use of an iron-score based on the expression level of at least 1 gene, in particular at least 3, preferably at least 5, and even preferably 8 genes selected in the group consisting of APEX1, TFRC, HIF1A, ABCG2, SCARA3, IREB2, SFXN4 and SLC39A14 involved in the iron metabolism, as a prognosis marker in subjects having MCL, in particular for identifying subjects with a poor outcome such as a relapse and/or death.

### FIELD OF THE INVENTION

The present invention relates to the field of in vitro method for prognosing the outcome of a subject affected by MCL, as well as associated therapeutic uses and methods.

### BACKGROUND OF THE INVENTION

Lymphomas can affect any organ in the body, present with a wide range of symptoms. They are traditionally divided into Hodgkin's lymphoma (which accounts for about 10% of all lymphomas) and non-Hodgkin lymphoma. Non-Hodgkin lymphoma represents a wide spectrum of illnesses that vary from the most indolent to the most aggressive malignancies. They arise from lymphocytes that are at various stages of development, and the characteristics of the specific lymphoma subtype reflect those of the cell from which they originated. The human mature B cell malignancies represent a medical challenge that is only partly met by current therapy, justifying concerted investigation into their molecular circuitry and pathogenesis. Each lymphoma subtype bears a phenotypic resemblance to B cells at a particular stage of differentiation, as judged by the presence or absence of immunoglobulin (Ig) variable (V) region mutations and by gene expression profiling.

The most common B-Cell lymphomas are non-Hodgkin lymphoma, in particular: Diffuse large B-cell lymphoma (DLBCL), Follicular lymphoma, Marginal zone B-cell lymphoma (MZL) or Mucosa-Associated Lymphatic Tissue lymphoma (MALT), Small lymphocytic lymphoma (also known as chronic lymphocytic leukemia, CLL), and Mantle cell lymphoma (MCL).

The present invention will focus as examples to MCL.

Mantle cell lymphoma (MCL) accounts for about 6% of all non-Hodgkin lymphomas (NHL). Median age at diagnosis is mid-60s, with a 3:1 male:female ratio and frequent extranodal involvement (bone marrow, blood, and gastrointestinal tract particularly). The median overall survival (OS) has improved, although for the overall population, median OS remains <3 years. Derived from mostly antigen-naive cells, MCL cells proliferate in the mantle zone around germinal centers (2), with morphologic (diffuse, nodular, mantle zone) as well as cytologic variants (small cells, pleomorphic, blastoid). Diagnosis suspected on immunophenotype requires confirmation by CYCLIN D1 overexpression due to t(11;14) translocation. Rare cases of Cyclin D1 negative MCL show Cyclin D2 or D3 overexpression and share similar clinical behavior and outcome with Cyclin D1-positive cases.

The inventors developed an Iron score for MCL subjects, which is a gene expression profile (GEP)-based risk score based on 8 prognostic genes. Iron plays a central role in a large number of essential cellular functions, including oxygen sensing, energy metabolism, respiration and folate metabolism, and is also required for cell proliferation, serving as a cofactor for several enzymes involved in DNA synthesis and DNA repair. The iron score of the present invention allows to identify MCL patients with a poor outcome and that could benefit from targeted therapy. In addition, the inventors demonstrated that Ironomycin, an iron chelator, significantly reduces the median number of viable primary MCL cells of patients without major toxicity for non-tumor cells from the microenvironment and presented a low toxicity on hematopoietic progenitors compared to conventional treatment. Interestingly, the inventors also identified a significant synergistic effect when Ironomycin is combined with Doxorubicin or with Ibrutinib (BTK inhibitor) or with Venetoclax (Bcl2 Inhibitor).

Altogether, these data demonstrated that a subgroup of MCL patients could be identified with the iron score and could benefit from a treatment comprising an inhibitor of iron metabolism, in particular Ironomycin or AM23 alone or in combination with conventional MCL treatments.

### SUMMARY OF THE INVENTION

A first object of the present invention is the use of an iron-score based on the expression level of at least 1 gene, in particular at least 5, preferably at least 7, and even preferably 8 genes selected in the group consisting of APEX1, TFRC, HIF1A, ABCG2, SCARA3, IREB2, SFXN4 and SLC39A14 involved in the iron metabolism, as a prognosis marker in subjects having MCL, in particular for identifying subjects with a poor outcome such as a relapse and/or death.

In a particular embodiment, the present invention concerns the use of an iron-score based on the expression level of at least 3, preferably at least 5, and even preferably 8 genes and/or proteins encoded by the said at least 3, preferably at least 5, and even preferably 8 genes selected in the group consisting of APEX1, TFRC, HIF1A, ABCG2, SCARA3, IREB2, SFXN4 and SLC39A14 involved in the iron metabolism, as a prognosis marker in MCL subjects, in particular for identifying MCL subjects with a poor outcome such as a relapse and/or death.

The invention also concerns an in vitro method for identifying MCL subject with a poor outcome that may benefit from a therapeutic treatment targeting iron metabolism, comprising the steps of:
a) Measuring the expression level of at least 1, in particular at least 3, preferably at least 5, and even preferably 8 genes and/or proteins encoded by the said at least 3, preferably at least 5, and even preferably 8 genes selected in the group consisting of APEX1, TFRC, HIF1A, ABCG2, SCARA3, IREB2, SFXN4 and SLC39A14 involved in the iron metabolism, in a biological sample obtained from said subject;
b) Calculating a score value from said expression level obtained at step a)
c) Classifying and identifying the said subject as having a poor outcome according to the score value in comparison to a predetermined reference value.

In a particular embodiment, the present invention concerns an in vitro method for identifying MCL subjects with a poor outcome that may benefit of a therapeutic treatment targeting iron metabolism, comprising the steps of:
a) Measuring the expression level of at least 3, preferably at least 5, and even preferably 8 genes and/or proteins encoded by the said at least 3, preferably at least 5, and even preferably 8 genes selected in the group consisting of APEX1, TFRC, HIF1A, ABCG2, SCARA3, IREB2, SFXN4 and SLC39A14 involved in the iron metabolism, in a biological sample obtained from said subject;
b) Calculating a score value from said expression level obtained at step a)
c) Classifying and identifying the said subject with a poor outcome according to the score value in comparison to a predetermined reference value.

Another subject-matter of the present invention is an in vitro method for monitoring the efficacy of a therapeutic treatment targeting iron metabolism in a subject having MCL and undergoing said treatment, comprising the steps of:
a) Measuring the expression level of at least 1, in particular at least 3, preferably at least 5, and even preferably 8 genes and/or proteins encoded by the said at least 3, preferably at least 5, and even preferably 8 genes selected in the group consisting of APEX1, TFRC, HIF1A, ABCG2, SCARA3, IREB2, SFXN4 and SLC39A14 involved in the iron metabolism, in a biological sample obtained from said subject at a time T1 before or during or after the subject has been administered said therapeutic treatment targeting iron metabolism;
b) Calculating a score value at time T1 from said expression level obtained at step a)
c) Measuring the expression level of at least at least 1, in particular at least 3, preferably at least 5, and even preferably 8 genes and/or proteins encoded by the said at least 3, preferably at least 5, and even preferably 8 genes selected in the group consisting of APEX1, TFRC, HIF1A, ABCG2, SCARA3, IREB2, SFXN4 and SLC39A14 involved in the iron metabolism, in a biological sample obtained from said subject at a time T2 before or during or after the subject has been administered the said therapeutic treatment targeting iron metabolism, wherein said time T2 is posterior to said time T1;
d) Calculating a score value at time T2 from said expression level obtained at step c),
e) Assessing the efficacy of a therapeutic treatment based on the comparison the score value at T2 obtained at step d) with the score value at T1 obtained at step b).

In a particular embodiment, the invention concerns an in vitro method for monitoring the efficacy of a therapeutic treatment targeting iron metabolism in a subject having MCL and undergoing said treatment, comprising the steps of:
a) Measuring the expression level of at least 3, preferably at least 5, and even preferably 8 genes and/or proteins encoded by the said at least 3, preferably at least 5, and even preferably 8 genes selected in the group consisting of APEX1, TFRC, HIF1A, ABCG2, SCARA3, IREB2, SFXN4 and SLC39A14 involved in the iron metabolism, in a biological sample obtained from said subject at a time T1 before or during or after the subject has been administered said therapeutic treatment targeting iron metabolism;
b) Calculating a score value at time T1 from said expression level obtained at step a)
c) Measuring the expression level of at least at least 3, preferably at least 5, and even preferably 8 genes and/or proteins encoded by the said at least 3, preferably at least 5, and even preferably 8 genes selected in the group consisting of APEX1, TFRC, HIF1A, ABCG2, SCARA3, IREB2, SFXN4 and SLC39A14 involved in the iron metabolism, in a biological sample obtained from said subject at a time T2 before or during or after the subject has been administered the said therapeutic treatment targeting iron metabolism, wherein said time T2 is posterior to said time T1;
d) Calculating a score value at time T2 from said expression level obtained at step c),
e) Assessing the efficacy of a therapeutic treatment based on the comparison the score value at T2 obtained at step d) with the score value at T1 obtained at step b).

The in vitro methods of the present invention optionally comprise one or more housekeeping gene(s) for normalization of the data.

By “housekeeping genes”, it is meant genes that are constitutively expressed at a relatively constant level across many or all known conditions, because they code for proteins that are constantly required by the cell, hence, they are essential to a cell and always present under any conditions. It is assumed that their expression is unaffected by experimental conditions. The proteins they code are generally involved in the basic functions necessary for the sustenance or maintenance of the cell. Non-limitating examples of housekeeping genes that may be used in methods of the invention include:
HPRT1 (hypoxanthine phosphoribosyltransferase 1),
UBC (ubiquitin C),
YWHAZ (tyrosine 3-monooxygenase/tryptophan 5-monooxygenase activation protein, zeta polypeptide),
B2M (beta-2-microglobulin),
GAPDH (glyceraldehyde-3-phosphate dehydrogenase),
FPGS (folylpolyglutamate synthase),
DECR1 (2,4-dienoyl CoA reductase 1, mitochondrial),
PPIB (peptidylprolyl isomerase B (cyclophilin B)),
ACTB (actin β),
PSMB2 (proteasome (prosome, macropain) subunit, beta type, 2),
GPS1 (G protein pathway suppressor 1),
CANX (calnexin),
NACA (nascent polypeptide-associated complex alpha subunit),
TAX1BP1 (Taxi (human T-cell leukemia virus type I) binding protein 1), and
PSMD2 (proteasome (prosome, macropain) 26S subunit, non-ATPase, 2).

When such housekeeping genes are added to the expression profile (it is not always necessary), they are used for normalization purpose. In this case, the number of housekeeping genes used for normalization in methods according to the invention is preferably comprised between one and five with a preference for three.

The in vitro methods of the present invention comprise a step of measuring the expression level of at least 1, 2, 3, 4, 5, 6, 7, or 8 genes useful for the outcome prognostic, also named ‘prognosis genes’ or genes of interest’ according to the invention.

The present invention also relates to a kit dedicated to in vitro methods according to the invention, in particular for MCL subjects, comprising or consisting of reagents for determining the expression level of at least 1, preferably at least 3, more preferably at least 5 and even preferably at least 8 genes and/or proteins selected in the group consisting of APEX1, TFRC, HIF1A, ABCG2, SCARA3, IREB2, SFXN4 and SLC39A14 in a sample of said subject.

The invention also relates to a pharmaceutical composition comprising, in a pharmaceutical acceptable vehicle, an molecule targeting iron metabolism in particular iron chelators and small molecules sequestering lysosomal iron, in particular selected in the group consisting of Deferasirox, Deferoxamine, Deferiprone, Salinomycin, analogs or derivatives thereof, preferably salinomycin and nitrogen-containing analogs of salinomycin, for use in a method for treating subjects having Mantle cell lymphoma (MCL).

In a particular embodiment, the pharmaceutical composition is used in a method for treating MCL subjects identified according to the in vitro method of the invention as having a poor outcome according to iron-score and consequently likely to display MCL relapse and/or death.

Another subject-matter of the invention is a pharmaceutical product comprising:
(i) a molecule targeting iron metabolism, in particular an iron chelator or small molecule sequestering lysosomal iron and
(ii) another anti-cancer agents selected from the group consisting of agents used in chemotherapy, targeted treatments, immune therapies, and combinations thereof,
   as combination product for simultaneous, separate or staggered use as a medicament in the treatment of MCL, in particular in MCL subjects with a poor outcome according to in vitro method of the invention.

The present invention also relates to systems (and computer readable medium for causing computer systems) to perform the in vitro methods of the invention, based on above described expression levels of genes and/or proteins as identified above.

In particular, the system includes a machine-readable memory, such as a computer or/and a calculator, and a processor configured to compute R Maxstat function and Cox multivariate function, according to the invention. This system is dedicated to perform the in vitro methods according to the invention in particular for identifying B-Cell lymphoma subjects with a poor outcome.

In particular, the system 1 for analyzing a biological sample of a subject affected by MCL comprises:
(a) a determination module 2 configured to receive a biological sample and to determine expression level information concerning the prognosis genes as disclosed in the present invention and optionally one or more housekeeping gene(s);
(b) a storage device 3 configured to store the expression level information from the determination module;
(c) a comparison module 4 adapted to compare the expression level information stored on the storage device with reference data, and to provide a comparison result, wherein the comparison result is indicative of the outcome of the subject; and
(d) a display module 5 for displaying a content based in part on the comparison result for the user, wherein the content is a signal indicative of the outcome of the subject.

### Definitions

The term ‘subject’ or ‘patient’ or ‘individual’ refers to a human subject, whatever its age or sex. The subject is affected by a B-Cell Lymphoma, in particular MCL. The subject may be already subjected to a treatment, by any chemotherapeutic agent, or may be untreated yet.

The term ‘MCL subject’ refers to a subject having MCL originating from a population of MCL subjects, from early to late stage of MCL, the said subjects undergoing or not undergoing a therapeutic treatment, and in particular MCL subjects experiencing relapsing MCL.

The ‘iron-score’ according to the invention is a GEP (Gene Expression Profile)-based iron-score; it is defined by the sum of the beta coefficients of the Cox model for each prognostic gene, weighted by ±1 according to the patient signal above or below the probe set Maxstat value.

By ‘prognosis marker’, it means a marker relevant to assess the outcome of the subject. In particular the expression profile or expression level of 1 to 8 genes and/or proteins identified in the present invention as being differentially expressed in MCL subjects, represents a prognosis marker that permits to identify subjects having ‘good prognosis’ from subjects having ‘bad prognosis’.

The 8 genes for MCL identified to be informative to assess the outcome of the subject are also named in the disclosure as ‘genes of interest’ or ‘prognosis genes’ or ‘prognostic genes’.

By ‘good prognosis’ or ‘good outcome’ according to the present invention, it means the survival of the subject.

By ‘poor prognosis’ or ‘poor outcome’ according to the present invention, it means the ‘disease relapse’ or the ‘death’ of the subject.

By ‘therapeutic treatment targeting iron metabolism’ according to the invention, it encompasses iron chelators and small molecules that sequester lysosomal iron. Examples of such molecules are illustrated later in the disclosure.

The term “treating” or “treatment” means stabilizing, alleviating, curing, or reducing the progression of MCL.

A ‘biological sample’ according to the invention refers to a biological sample obtained, isolated or collected from a subject, in particular a cell culture, a cell line, a tissue biopsy or a fluid such as a blood or bone marrow. In particular, the biological sample is a tissue biopsy comprising lymph nodes or spleen or a fluid comprising lymphocytes B like blood or bone marrow.

By a “reference sample”, it is meant a biological sample of a patient whose clinical outcome is known (i.e. the duration of the disease-free survival (DFS), or the event free survival (EFS) or the overall survival (OS) or both). Preferably, a pool of reference samples comprises at least one (preferably several, more preferably at least 5, more preferably at least 6, at least 7, at least 8, at least 9, at least 10) ‘good outcome’ patient(s) and at least one (preferably several, more preferably at least 6, at least 7, at least 8, at least 9, at least 10) ‘bad outcome’ patient(s). The highest the number of reference samples, the better for the reliability of the method of prediction of the outcome of the subject tested according to the invention.

Said reference samples (collection samples of B-Cell Lymphoma subjects) for which expression profile of the prognosis genes is evaluated, permits to measure predetermined reference values (PREV and PREL as further disclosed), which are used for comparison purposes.

### BRIEF DESCRIPTION OF THE FIGURES

FIG. 1: Prognostic value of the Iron-score in MCL patients.

Patients of the Staudt cohort GSE10793 (n=71) were ranked according to increased iron score and a maximum difference in OS (overall survival) was obtained with iron score of −3.7798 (also named ‘cut point’) splitting patients into high-risk and low-risk groups. The iron score was significantly associated with high-risk in MCL patients.

FIG. 2: Ironomycin kills MCL cells with nanomolar concentration

The panel of 6 MCL cell lines were incubated with increasing concentrations of Ironomycin (A), AM23 (B) or vehicle for 96H.

FIG. 3: Apoptosis induced by Ironomycin was not reversed by iron supplementation

Jeko-1 and JVM2 cell line was pre-incubated with or without 80 μM of deferasirox or 50 nM and 500 nM of Ironomycin, respectively, for 4 hours followed by 72H incubation in presence or absence of FeCl3 (100 μM). Apoptosis was assessed using Annexin V-PE staining by flow cytometry. Iron supplementation significantly inhibited the effect of iron chelators on MCL cells apoptosis (P<0.05 and P<0.01 for Deferasirox treatment). However, iron supplementation did not affect ironomycin-induced MCL cell cytotoxicity.

FIG. 4: Ironomycin induces MCL cell cycle defect.

Cells were incubated with vehicle or with IC₅₀ Ironomycin for 24 hours. Cell cycle was analyzed using flow cytometry, S phase was stained by an anti-BrdU antibody after BrdU incorporation and DNA content was strained by 4′,6-diamidino-2-phénylindole (DAPI) for Jeko-1 and JVM-2 cell lines. Histograms represent the mean percentage and SD of each cell cycle phase of three independent experiments. * and ** indicate a significant difference of P<0.05 and P<0.01, respectively with paired student t-test.

FIG. 5: Ironomycin induces DNA damage response: double strand breaks evidenced by Serine 139 phosphorylation of histone variant H2A.X.

Cells were treated with Ironomycin (100 nM for Jeko-1 and 500 nM for JVM-2) during 24H. Protein levels of Phospho-H2A.X (S139) were analyzed by western blot and normalized by β-actin protein level.

FIG. 6: Ironomycin induces a significant downregulation of Cyclin D1 in MCL cell lines. Jeko-1 and JVM2 cells were treated with Ironomycin (100 nM and 500 nM, respectively) during 24H. Protein levels of cyclinD1, phospho-Rb, Rb and CDK4 were analyzed by western blot and normalized by the β-actin expression level. This Cyclin D1 downregulation is associated with a downregulation of Rb phosphorylation and CDK4 protein levels.

FIG. 7: Assays on primary MCL cells of patients

In a first experiment, primary MCL cells were treated with Ironomycin and incubated during 96H with CD40L. The toxicity on MCL cells (A) and non-MCL cells (B) was analyzed by flow cytometry and expressed in % of control. N=5, Median+/−IQR, t-test of pairs. * P<0.05, ** P<0.01, *** P<0.001, **** P<0.0001.

These first results were further completed by additional experiments: primary MCL cell were treated with Ironomycin (C) or AM-23 (D) and incubated during 96H with recombinant CD40L. Tumorous cells were analyzed by flow cytometry (described in material and method) and expressed in % of control.

Results represent the median±IQR of each population cells of nine (C) and six patients (D), respectively. Statistical significance was tested using t-test of pairs: * P<0.05, ** P<0.01 *** P<0.001, **** P<0.0001 and NS: non-significant.

FIG. 8: Synergistic effect of Ironomycin with Ibrutinib BTK inhibitor.

Jeko-1 and JVM-2 cells were treated with increasing concentrations of ironomycin combined with ibrutinib (BTK inhibitor) for 96 h and cell viability was tested by ATP quantification to obtain the viability matrix. The synergy matrix was calculated as described in Material and Methods.

FIG. 9: Synergistic effect of Ironomycin with Venetoclax Bcl2 inhibitor.

Jeko-1 and JVM-2 cells were treated with increasing concentrations of ironomycin combined with Venetoclax (BCL2 inhibitor) for 96 h and cell viability was tested by ATP quantification to obtain the viability matrix. The synergy matrix was calculated as described in Material and Methods.

FIG. 10: Synergistic effect of Ironomycin with Doxorubicin.

Jeko-1 and JVM-2 cells were treated with increasing concentrations of ironomycin combined with doxorubicin for 96 h and cell viability was tested by ATP quantification to obtain the viability matrix. The synergy matrix was calculated as described in Material and Methods.

### DETAILED DESCRIPTION OF EMBODIMENTS OF THE INVENTION

The inventors have identified a set of 8 genes and/or proteins involved in the iron metabolism, which are differentially expressed in individuals having MCL (MCL cells) as compared to healthy subjects (normal B cells). This gene expression profile (GEP)-based risk score may be advantageously used for identifying subjects with poor outcome that may benefit of a targeted treatment (also named personalized medicine) comprising an iron inhibitor. A score value has been calculated, taking into account the beta coefficient for each gene or protein, based on the Cox statistical model.

As illustrated in the examples, the inventors identified, from a list of 63 genes involved in the regulation of iron biology and using Maxstat R function and Benjamini Hochberg multiple testing correction, 8 genes demonstrated a prognostic value in a cohort of MCL patients (n=71).

In particular, the inventors demonstrated that:
high expression of four genes was associated with a good prognosis (‘good outcome’) including ABCG2 (ATP-binding cassette transporter G2), SCARA3 (Scavenger Receptor Class A Member 3), IREB2 (Iron Responsive Element Binding Protein 2) and SFXN4 (sideroflexin 4); and

high expression of four genes was associated with a poor prognosis (‘poor outcome’): APEX1 (DNA-(apurinic or apyrimidinic site) lyase), TFRC (Transferrin Receptor Protein 1), SLC39A14 (Solute Carrier Family 39 Member 14), and HIF1A (Hypoxia inductible factor A 1). So the present invention concerns the use of an iron-score based on the expression level of at least 1 gene, in particular at least 3, preferably at least 5, and even preferably 8 genes and/or proteins encoded by the said at least 3, preferably at least 5, and even preferably 8 genes selected in the group consisting of APEX1, TFRC, HIF1A, ABCG2, SCARA3, IREB2, SFXN4 and SLC39A14 involved in the iron metabolism, as a prognosis marker in subjects having MCL, in particular for identifying subjects with a poor outcome such as a relapse and/or death.

Such MCL subjects with a poor outcome such as a relapse and/or death identified according to the invention by their iron-score value, may be advantageously treated by a targeted therapeutic treatment comprising an inhibitor of iron metabolism.

In a particular embodiment, the said targeted therapeutic treatment comprises a molecule targeting iron metabolism in particular iron chelator or small molecule sequestering lysosomal iron, in particular selected in the group consisting of Deferasirox, Deferoxamine, Deferiprone, Salinomycin, analogs or derivatives thereof, preferably salinomycin and nitrogen containing salinomycin derivatives.

By ‘molecule targeting iron metabolism’ according to the invention, it means in particular iron chelators and small molecules sequestering lysosomal iron. Iron chelators are small molecules susceptible to interact reversibly with iron. And small molecules sequestering lysosomal iron are loose iron binders that accumulate in the endosomal/lysosomal compartment able to block the metal in this organelle. Examples of such compounds are disclosed later in the description.

By ‘derivatives thereof’ according to the invention, it means synthetic small molecules chemically derived from salinomycin exhibiting a more potent activity and potentially lower toxicity against healthy cells.

By ‘at least 1, in particular at least 3’ genes and/or proteins, it means 1, 2, 3, 4, in particular 5, 6, 7, 8 genes, or 1, 2, 3, 4, in particular 5, 6, 7, 8, proteins.

In an embodiment, the combination of 2 genes and/or proteins encoded by the said genes selected in the group consisting of APEX1, TFRC, HIF1A, ABCG2, SCARA3, IREB2, SFXN4 and SLC39A14 is evaluated.

In an embodiment, the combination of 3 genes and/or proteins encoded by the said genes selected in the group consisting of APEX1, TFRC, HIF1A, ABCG2, SCARA3, IREB2, SFXN4 and SLC39A14 is evaluated.

In an embodiment, the combination of 4 genes and/or proteins encoded by the said genes selected in the group consisting of APEX1, TFRC, HIF1A, ABCG2, SCARA3, IREB2, SFXN4 and SLC39A14 is evaluated.

In an embodiment, the combination of 5 genes and/or proteins encoded by the said genes selected in the group consisting of APEX1, TFRC, HIF1A, ABCG2, SCARA3, IREB2, SFXN4 and SLC39A14, is evaluated.

In another embodiment, the combination of 6 genes and/or proteins encoded by the said genes selected in the group consisting of APEX1, TFRC, HIF1A, ABCG2, SCARA3, IREB2, SFXN4 and SLC39A14, is evaluated.

In another embodiment, the combination of 7 genes and/or proteins encoded by the said genes selected in the group consisting of APEX1, TFRC, HIF1A, ABCG2, SCARA3, IREB2, SFXN4 and SLC39A14, is evaluated.

In another embodiment, the combination of 8 genes and/or proteins encoded by the said genes selected in the group consisting of APEX1, TFRC, HIF1A, ABCG2, SCARA3, IREB2, SFXN4 and SLC39A14, is evaluated.

The NCBI references for each gene are mentioned in the table 1 hereunder:

TABLE 1

| | | Ref Seq | Ref Seq | |
|---|---|---|---|---|
| Gene | | Transcript ID | Protein ID | |
| symbol | Gene Name | (NCBI) | (NCBI) | Gene ID |
| APEX1 | DNA-(apurinic or | NM_001641 | NP_001632.2 | 328 |
| | apyrimidinic site) lyase | | | |
| TFRC | Transferrin Receptor | NM_003234 | NP_003225.2 | 7037 |
| | Protein 1 | | | |
| ABCG2 | ATP-binding cassette | NM_004827 | NP_004818.2 | 9429 |
| | transporter G2 | | | |
| SCARA3 | Scavenger Receptor | NM_016240 | NP_057324.2 | 51435 |
| | Class A Member 3 | | | |
| IREB2 | Iron Responsive Element | NM_004136 | NP_004127.2 | 3658 |
| | Binding Protein 2 | | | |
| SLC39A14 | Solute Carrier Family 39 | NM_015359 | NP_056174.2 | 23516 |
| | Member 14 | | | |
| SFXN4 | sideroflexin 4 | NM_178867 | NP_849198.2 | 119559 |
| HIF1A | Hypoxia inductible factor | NM_001530 | NP_001521.1 | 3091 |
| | A 1 | | | |

Expression Level of the Set of the Said Genes or Proteins of Interest (‘Prognosis Genes’)

Such measures are made in vitro, starting from a subject's sample, and necessary involve transformation of the sample. Indeed, no measure of a specific gene expression level can be made without some type of transformation of the sample. Most technologies rely on the use of reagents specifically binding to the RNA of interest, thus resulting in a modified sample further including the detection reagent. In addition, most technologies also involve some preliminary extraction of RNA from the subject's sample before binding to a specific reagent. The claimed method may thus also comprise a preliminary step of extracting RNA from the subject's sample.

The expression level of the set of genes and/or proteins, in particular selected in the group consisting of APEX1, TFRC, HIF1A, ABCG2, SCARA3, IREB2, SFXN4 and SLC39A14, according to the invention, may be measured by any techniques commonly used. The presence or level of said genes is determined by usual method known from man skilled in the art. In particular, each gene expression level may be measured at the genomic and/or nucleic and/or protein level. In a preferred embodiment, the expression profile is determined by measuring the amount of nucleic acid transcripts of each gene, such as PCR, quantitative PCR (qPCR), NGS (Next-Generation Sequencing (NGS)) and RNA sequencing. In another embodiment, the expression profile is determined by measuring the amount of protein produced by each of the genes.

The amount of nucleic acid transcripts can be measured by any technology known by a man skilled in the art. In particular, the measure may be carried out directly on an extracted messenger RNA (mRNA) sample, or on retrotranscribed complementary DNA (cDNA) prepared from extracted mRNA by technologies well-known in the art. From the mRNA or cDNA sample, the amount of nucleic acid transcripts may be measured using any technology known by a man skilled in the art, including nucleic microarrays, quantitative PCR, next generation sequencing and hybridization with a labelled probe.

PCR primers for the DNA amplicons encompassing the genes of interest disclosed above were designed using the genomic sequence obtained from the NCBI.

In particular, the level of mRNA expression for each of the genes of the set may be performed by the well-known techniques of the skilled in the art such as hybridization technique and/or amplification technique (PCR), using suitable primers or probes that are specific for each of the genes mRNA.

Illustratively, mRNA may be extracted, for example using lytic enzymes or chemical solutions or extracted by commercially available nucleic-acid-binding resins following the manufacturer's instructions. Extracted mRNA may be subsequently detected by hybridization, such as Northern blot, and/or amplification, such as quantitative or semiquantitative RT-PCR. Other methods of amplification include ligase chain reaction (LCR), transcription-mediated amplification (TMA), strand displacement amplification (SDA) and nucleic acid sequence based amplification (NASBA). In some embodiments, the level of mRNA expression for each of the genes of interest may be measured by the mean of quantification of the cDNA synthesized from said mRNA, as a template, by one reverse transcriptase.

The amount of mRNA can be measured by any technology known by a person skilled in the art, including mRNA microarrays, quantitative PCR, next generation sequencing and hybridization with a labelled probe. In particular, real time quantitative RT-PCR (qRT-PCR) may be useful. In some embodiments, qRT-PCR can be used for both the detection and quantification of RNA targets. Commercially available qRT-PCR based methods (e.g., Taqman® Array) may for instance be employed, the design of primers and/or probe being easily made based on the sequence of ‘prognostic genes’ disclosed above. mRNA assays or arrays can also be used to assess the levels of the mRNAs in a sample. In some embodiments, mRNA oligonucleotide array can be prepared or purchased. An array typically contains a solid support and at least one oligonucleotide contacting the support, where the oligonucleotide corresponds to at least a portion of a mRNA.

Any suitable assay platform can be used to determine the presence of the mRNA in a sample. For example, an assay may be in the form of a membrane, a chip, a disk, a test strip, a filter, a microsphere, a multiwell plate, and the like. An assay system may have a solid support on which an oligonucleotide corresponding to the mRNA is attached. The solid support may comprise, for example, a plastic, silicon, a metal, a resin, or a glass. The assay components can be prepared and packaged together as a kit for detecting an mRNA. To determine the expression profile of a target nucleic sample, said sample is labelled, contacted with the microarray in hybridization conditions, leading to the formation of complexes between target nucleic acids that are complementary to probe sequences attached to the microarray surface. The presence of labelled hybridized complexes is then detected. Many variants of the microarray hybridization technology are available to the person skilled in the art.

Methods for determining the quantity of mRNA by microarrays or by RNA sequencing may also be used. In certain embodiments, complexes between the double-stranded nucleic acids resulting from amplification and fluorescent SYBR® molecules may be obtained and then the fluorescence signal generated by the SYBR® molecules complexed with the said amplified nucleic acids may be measured. Identification of suitable primers that are specific for each of the genes mRNA consists of a routine work for the one skilled in the art.

In a particular embodiment and as illustrated in the examples for MCL subjects, the method for determining the quantity of mRNA by microarrays uses probesets for the specific 8 prognostic genes disclosed above. Mention may be made of the Lymphochip cDNA microarray and probesets ID related to said specific 8 prognostic genes. In a particular embodiment, method for determining the quantity of mRNA by microarrays uses 8 probesets for the specific 8 prognostic genes, as illustrated in the further examples.

In some embodiments, detection by hybridization may be performed with a detectable lable, such as fluorescent probes, enzymatic reactions or other ligands (eg avidin/biotin).

The presence or level of said proteins may be measured by well-known techniques including detection and quantification of the protein of interest by the means of any type of ligand molecule that specifically binds thereto, including nucleic acids (for example nucleic acids selected for binding through the well-known SELEX method), antibodies and antibody fragments. The antibodies to said given protein of interest may be easily obtained with the conventional techniques, including generation of antibody-producing hybridomas.

Thus, in preferred embodiments, expression of a marker is assessed using for example:
a radio-labelled antibody, in particular, a radioactive moiety suitable for the invention may for example be selected within the group comprising 3H, 121I, 123I, 14C or 32P;
a chromophore-labelled or a fluorophore-labelled antibody, wherein a luminescent marker, and in particular a fluorescent marker, suitable for the invention may be any marker commonly used in the field such as fluorescein, fluorescent probes, coumarin and its derivatives, phycoerythrin and its derivatives, or fluorescent proteins such as GFP or the DsRed;
a polymer-backbone-antibody;
an enzyme-labelled antibody, said labelling enzyme suitable for the invention may be an alkaline phosphatase, a tyrosinase, a peroxydase, or a glucosidase; for example, suitable avidin-labelled enzyme may be an avidin-Horse Radish Peroxydase (HRP), and a suitable substrate may be AEC, 5-bromo-4-chloro-3-indolyl phosphate (BCIP), nitro blue tetrazolium chloride (NBT);
an antibody derivative, for example an antibody conjugated with a substrate or with the protein or ligand of a protein-ligand pair, in particular a biotin, a streptavidin or an antibody binding the polyhistidine tag;
an antibody fragment, for example a single-chain antibody, an isolated antibody hypervariable domain, etc., which binds specifically to a marker protein or a fragment thereof, including a marker protein which has undergone all or a portion of its normal post-translational modification.

In a particular and preferred embodiment, expression of a marker is assessed using a GFP fluorescent protein.

In vitro techniques for detection of a biological marker protein include enzyme linked immunosorbent assays (ELISAs), Western blots, immunoprecipitations and immunofluorescence.

In a particular and preferred embodiment, the preferred in vitro methods for detecting and quantifying level expression of said genes of interest according to the present invention, include micro-arrays, NGS, RNA sequencing and PCR techniques.

Calculation of a Score Value (‘Iron Score’) from Said Expression Level of Genes or Proteins of Interest

The score value or ‘prognosis score’ or ‘iron score’ according to the invention, based on the expression level of the ‘prognosis genes’ as defined above, will help classifying the B-Cell lymphoma subjects as having a ‘good outcome’ or a ‘bad outcome’.

The lower the expression of genes related to ‘bad outcome’ is, the better for the subject's survival. Therefore, the higher the level of iron score is, the more likely the subject is to respond to a treatment targeting iron metabolism. In a preferred embodiment, the subject may thus be predicted as having ‘poor outcome’ and consequently being likely to respond to a treatment targeting iron metabolism based on comparison of the expression level of said prognosis genes in the patient's sample with one or more threshold value(s) (predetermined reference value, PREV).

In a particular embodiment, the patient is considered as having poor outcome, when the iron score is higher than a threshold value. Such a threshold value may be determined based on a pool of reference samples, as defined above. In this embodiment, patients are classified into two groups based on said expression level of prognosis genes, depending if this expression level is lower or greater than said threshold value. Patients with iron score higher than the threshold value are considered as having a poor outcome and likely to respond to treatment targeting iron metabolism.

In another embodiment, the method further comprises determining a prognostic score based on the expression level of said prognosis genes, wherein the prognostic score indicates whether the patient has a poor outcome. In particular, said prognosis score may indicate whether the patient is likely to have a poor outcome or a bad outcome if it is higher or lower than a predetermined threshold value (PREV or PREL) (dichotomized result).

As a result, a prognosis score may be determined based on the analysis of the correlation between the expression level of said prognosis genes of the invention and progression free survival (PFS) or overall survival (OS) of a pool of reference samples, as defined above. A PFS and/or OS score, which is a function correlating PFS or OS to the expression level of said prognosis genes of the invention, may thus be used as prognosis score for prediction of the outcome of the subject.

The expression level for each combination of the 11 genes and/or proteins of interest as disclosed above according to the invention, may be associated with a score value, also named ‘iron-score’ in the present invention.

Following the measurement of the expression level of at least 1, in particular at least 3 or more genes and/or proteins encoded by the said 3 or more genes selected in a group consisting of APEX1, TFRC, HIF1A, ABCG2, SCARA3, IREB2, SFXN4 and SLC39A14, in a biological sample obtained from a MCL subject (step a) of the method), the computation of a score value may be performed by a method comprising the following steps:

i) comparing the expression level determined at step a) with a predetermined reference expression level (PREL);

ii) calculating the score value (‘iron score’) with the following formula:

$$Score = {\sum }_{i = 1}^{n} β i \times Ci$$

wherein
n represents the number of genes and/or protein which expression level is measured, i.e. n being comprised from 1 to 8, in particular from 3 to 8,
βi represents the regression β coefficient reference value for a given gene or protein, and
Ci represents “1” if the expression level of said gene or protein is higher than the predetermined reference level (PREL) or Ci represents “−1” if the expression level of the gene or the protein is lower than or equal to the predetermined reference level (PREL).

The predetermined reference level (PREL) is often referred as to “maxstat value” or “maxstat cutpoint”.

In some embodiments, a good prognosis status or ‘good outcome’ refers to an individual having a score value lower than or equal to a predetermined reference value (PRV).

In some embodiments, a bad prognosis status or ‘bad outcome’ refers to an individual having a score value higher than a predetermined reference value (PRV).

The “regression β coefficient reference value” may be easily determined by the skilled man in the art for each gene or protein using the well-known statistical Cox model, which is based on a modelling approach to analyze survival data. The purpose of the model is to simultaneously explore the effects of several variables on survival. When it is used to analyze the survival of patients in a clinical trial, the model allows isolating the effects of the treatment from the effects of other variables. The Cox model may also be referred as to proportional hazards regression analysis. In particular, this model is a regression analysis of the survival times (or more specifically, the so-called “hazard function”) with respect to defined variables. The “hazard function” is the probability that an individual will experience an event, e.g. death, within a small time interval, given that the individual has survived up to the beginning of the interval. It can therefore be interpreted as the risk of dying at time t. The quantity h0 (t) is the baseline or underlying hazard function and corresponds to the probability of dying (or reaching an event) when all the defined variables are zero. The baseline hazard function is analogous to the intercept in ordinary regression (since exp0=1). The “regression coefficient β” gives the proportional change that can be expected in the hazard, related to changes in the defined variables. The coefficient β is estimated by a statistical method called maximum likelihood. In survival analysis, the hazard ratio (HR) (Hazard Ratio=exp(β)) is the ratio of the hazard rates corresponding to the conditions described by two sets of defined variables.

Predetermined reference values, such as PREL or PRV, which are used for comparison purposes may consist of “cut-off” values.

For example, each reference (“cut-off”) value PREL for each gene or protein may be determined by carrying out a method comprising the following steps:

a) providing a collection of samples from subjects (patients) suffering from MCL (‘reference samples’);

b) determining the expression level of the relevant gene or protein for each sample contained in the collection provided at step a);

c) ranking the samples according to said expression level;

d) classifying said samples in pairs of subsets of increasing, respectively decreasing, number of members ranked according to their expression level;

e) providing, for each sample provided at step a), information relating to the actual clinical outcome for the corresponding MCL patient (i.e. the duration of the disease-free survival (DFS), or the event free survival (EFS) or the overall survival (OS) or both);

f) for each pair of subsets of tumor tissue samples, obtaining a Kaplan Meier percentage of survival curve;

g) for each pair of subsets of tumor tissue samples calculating the statistical significance (p value) between both subsets;

h) selecting as reference value PREL for the expression level, the value of expression level for which the p value is the smallest.

As an illustration, the expression level of a gene or a protein of interest may be assessed for 100 samples (‘reference samples’) of 100 subjects (patients). The 100 samples are ranked according to the expression level of said given gene or protein. Sample 1 may have the highest expression level and sample 100 may have the lowest expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding MCL patient, Kaplan Meier curves may be prepared for each of the 99 groups of two subsets. Also for each of the 99 groups, the p value between both subsets was calculated. The reference value PREL is then selected such as the discrimination based on the criterion of the minimum p value is the strongest. In other words, the expression level corresponding to the boundary between both subsets for which the p value is minimum is considered as the reference value. It should be noted that according to the experiments made by the inventors, the reference value PREL is not necessarily the median value of expression levels.

The skilled in the art also understands that the same technique of assessment of the PRV could be used for obtaining the reference value and thereafter for assessment of the response to the targeted treatment of the present invention comprising an inhibitor of iron metabolism. However in one embodiment, the reference value PRV is the median value of PRV.

As illustrated further in the examples of the invention, the prognostic information of these 8 genes of interest (‘prognosis genes’) was then combined in a GEP (Gene Expression Profile)-based iron-score. The ‘iron score’ is defined by the sum of the beta coefficients of the Cox model for each prognostic gene, weighted by ±1 according to the patient signal above or below the probe set Maxstat value as previously described (Herviou et al., 2018). Patients were ranked according to increased prognostic score and for a given score value (−3.7798), the difference in survival of patients with a prognostic score ≤−3.7798 or >−3.7798 was computed using Maxstat analysis (Moreaux et al. MCT 2012; BJC 2013).

In particular embodiment, the regression 13 coefficient reference value, the hazard ratio and the reference value PREP for each of the 8 genes or proteins of interest were measured. These values were measured on references samples of MCL subjects (>200 samples) but may vary from 5 to 15% depending of the number of reference samples. The highest the number of reference samples, the better for the reliability of the method of prediction of the outcome of the subject tested according to the invention.

The Table 2 below illustrates relevant parameter ranges for Maxstat_Cutpoint and beta coefficient for each of the 8 genes of interest.

TABLE 2

| Name | Maxstat_Cutpoint | P.logRank | beta coef |
|---|---|---|---|
| APEX1 | −0.41317 | 0.00759721 | 0.78756622 |
| TFRC | −0.9998 | 0.00876336 | 0.63448695 |
| ABCG2 | 0.11395 | 0.0087612 | −1.1118334 |
| SCARA3 | 0.71554 | 0.02759528 | −0.6673228 |
| IREB2 | −0.97304 | 0.03725952 | −0.7971817 |
| SLC39A14 | −1.41889 | 1.78E−08 | 1.59091193 |
| SFXN4 | −0.40645 | 0.00014255 | −1.2292853 |
| HIF1A | 0.4644 | 0.03050855 | 0.81516043 |

The score may be generated by a computer program and may be used in the in vitro method according to the invention in particular for identifying a MCL subject with a poor outcome that may benefit of a targeted treatment comprising an inhibitor of iron metabolism, and/or for further monitoring the efficacy of a targeted therapeutic treatment.

Method for Identifying a MCL Subject with a Poor Outcome

The present invention also concerns an in vitro method for identifying a MCL subject with a poor outcome that may benefit from a targeted therapeutic treatment comprising an inhibitor of iron metabolism, comprising the steps of:
a) Measuring the expression level of at least 1, in particular at least 3, preferably at least 5, and even preferably 8 genes and/or proteins encoded by the said at least 3, preferably at least 5, and even preferably 8 genes selected in the group consisting of APEX1, TFRC, HIF1A, ABCG2, SCARA3, IREB2, SFXN4 and SLC39A14 involved in the iron metabolism, in a biological sample obtained from said subject;
b) Calculating a score value from said expression level obtained at step a)
c) Classifying and identifying the said subject with a poor outcome according to the score value in comparison to a predetermined reference value.

In a particular embodiment, the present invention concerns an in vitro method for identifying a MCL subject with a poor outcome that may benefit from a targeted therapeutic treatment comprising an inhibitor of iron metabolism, comprising the steps of:
a) Measuring the expression level of at least 3, preferably at least 5, and even preferably 8 genes and/or proteins encoded by the said at least 3, preferably at least 5, and even preferably 8 genes selected in the group consisting of APEX1, TFRC, HIF1A, ABCG2, SCARA3, IREB2, SFXN4 and SLC39A14 involved in the iron metabolism, in a biological sample obtained from said subject;
b) Calculating a score value from said expression level obtained at step a)
c) Classifying and identifying the said subject with a poor outcome according to the score value in comparison to a predetermined reference value.

The expression level of the said genes or proteins of interest at step a) are measured according to the detection and/or quantification methods well known in the art. Examples of such methods are disclosed above.

The calculation of the score value (‘iron score’) at step b) is made as disclosed above, in particular by:

i) comparing the expression level determined at step a) with a predetermined reference expression level (PREL);

ii) calculating the score value with the following formula:

$$Score = {\sum }_{i = 1}^{n} β i \times Ci$$

wherein
n represents the number of genes and/or protein which expression level is measured, i.e. n being comprised from 3 to 8,
βi represents the regression β coefficient reference value for a given gene or protein, and
Ci represents “1” if the expression level of said gene or protein is higher than the predetermined reference level (PREL) or Ci represents “−1” if the expression level of the gene or the protein is lower than or equal to the predetermined reference level (PREL).

The classification of the subject according to ‘good outcome’ subgroup and ‘bad outcome’ subgroup is based according to its iron-score value in comparison to a predetermined reference value (PRV).

In the present invention, a subject with a ‘poor outcome’ refers to an individual having a score value higher than a predetermined reference value (PRV).

In a particular embodiment, for MCL subjects, when the iron score is based on the expression level of the 8 genes or proteins consisting of APEX1, TFRC, HIF1A, ABCG2, SCARA3, IREB2, SFXN4 and SLC39A14, the predetermined reference value (PRV) or ‘cutpoint’ is −3.7798, meaning that in the step c) of the in vitro method described above, the subject with a poor outcome according to the iron score are the ones having an iron score value higher than −3.7798.

Method for Monitoring the Efficacy of a Targeted Therapeutic Treatment

Another object of the invention is an in vitro method for monitoring the efficacy of a therapeutic treatment targeting iron metabolism in a subject having MCL and undergoing said treatment, comprising the steps of:
a) Measuring the expression level of at least 1, in particular at least 3, preferably at least 5, and even preferably 8 genes and/or proteins encoded by the said at least 3, preferably at least 5, and even preferably 8 genes selected in the group consisting of APEX1, TFRC, HIF1A, ABCG2, SCARA3, IREB2, SFXN4 and SLC39A14 involved in the iron metabolism, in a biological sample obtained from said subject at a time T1 before or during or after the subject has been administered said therapeutic treatment targeting iron metabolism;
b) Calculating a first score value at time T1 from said expression level obtained at step a)
c) Measuring the expression level of at least at least 1, in particular at least 3, preferably at least 5, and even preferably 8 genes and/or proteins encoded by the said at least 3, preferably at least 5, and even preferably 8 genes selected in the group consisting of APEX1, TFRC, HIF1A, ABCG2, SCARA3, IREB2, SFXN4 and SLC39A14 involved in the iron metabolism, in a biological sample obtained from said subject at a time T2 before or during or after the subject has been administered the said therapeutic treatment targeting iron metabolism, wherein said time T2 is posterior to said time T1;
d) Calculating a second score value at time T2 from said expression level obtained at step c),
e) Assessing the efficacy of a therapeutic treatment based on the comparison the second score value at T2 obtained at step d) with the first score value at T1 obtained at step b).

The expression level of genes or proteins of interest according to the invention at step a) and d) are made as disclosed above.

The first and second score values (iron-score values), respectively at time T1 and time T2, are made as disclosed above.

In a preferred embodiment, the invention concerns an in vitro method for monitoring the efficacy of a therapeutic treatment targeting iron metabolism in a subject having MCL and undergoing said treatment, comprising the steps of:
a) Measuring the expression level of the 8 genes or proteins consisting of APEX1, TFRC, HIF1A, ABCG2, SCARA3, IREB2, SFXN4 and SLC39A14 involved in the iron metabolism, in a biological sample obtained from said subject at a time T1 before the subject has been administered said therapeutic treatment comprising an active agent against MCL and/or an inhibitor of iron metabolism;
b) Calculating a first score value at time T1 from said expression level obtained at step a)
c) Measuring the expression level of the 8 genes or proteins consisting of APEX1, TFRC, HIF1A, ABCG2, SCARA3, IREB2, SFXN4 and SLC39A14 involved in the iron metabolism, in a biological sample obtained from said subject at a time T2 after the subject has been administered the said therapeutic treatment comprising an active agent against MCL and/or an inhibitor of iron metabolism, wherein said time T2 is posterior to said time T1;

d) Calculating a second score value at time T2 from said expression level obtained at step c),
e) Assessing the efficacy of a therapeutic treatment based on the comparison the second score value at T2 obtained at step d) with the first score value at T1 obtained at step b).

Kits Dedicated for In Vitro Methods of the Invention

The kits of the invention are dedicated for in vitro methods of the invention.

By “dedicated”, it is meant that reagents for the determination of an expression level of genes and/or proteins as identified above in the kit of the invention essentially consist of reagents for determining the expression level of the above (i) expression profiles, optionally with one or more housekeeping gene(s), and thus comprise a minimum of reagents for determining the expression of other genes than those mentioned in above described (i) expression profiles and housekeeping genes. For instance, a dedicated kit of the invention preferably comprises no more than 20, preferably no more than 12, preferably no more than 10, preferably no more than 9, 8, 7, 6, 5, 4, 3, 2, or 1 reagent(s) for determining the expression level of a gene that does not belong to one of the above described (i) expression profiles and that is not a housekeeping gene.

Such a kit may further comprise instructions for determination of poor or good outcome of the subject.

So the present invention relates to a kit dedicated to in vitro methods of the invention, in particular for determining whether a MCL subject, has a high risk of death and/or relapse, comprising or consisting of reagents for determining the expression level of at least 1, preferably at least 3, more preferably at least 5 and even preferably at least 8 genes and/or proteins selected in the group consisting of APEX1, TFRC, HIF1A, ABCG2, SCARA3, IREB2, SFXN4 and SLC39A14 in a sample of said subject, and no more than 20, preferably no more than 12, preferably no more than 10, preferably no more than 9, 8, 7, 6, 5, 4, 3, 2, or 1 reagent(s) for determining the expression level of a gene that does not belong to one of the above described.

Reagents for determining the expression level of said prognostic genes in a sample of said subject, may notably comprise or consist of primers pairs (forward and reverse primers) and/or probes (in particular labeled probes, comprising a nucleic acid specific for the target sequence and a label attached thereto, in particular a fluorescent label) specific for said prognostic genes or a microarray comprising a sequence specific for said prognostic genes. The design of primers and/or probe can be easily made by those skilled in the art based on the sequences of said genes disclosed above.

In a particular embodiment, said kits comprise specific amplification primers and/or probes for the specific quantitative amplification of transcripts of ‘prognosis genes’ identified above and/or a nucleic microarray for the detection of said ‘prognosis genes’ identified above.

The present invention also relates to a kit dedicated to in vitro methods of the present invention comprising a set of primers and/or probes for measuring the expression level of at the least 3, preferably at least 5, and even preferably 8 genes and/or proteins encoded by the said at least 3, preferably at least 5, and even preferably 8 genes selected in the group consisting of APEX1, TFRC, HIF1A, ABCG2, SCARA3, IREB2, SFXN4 and SLC39A14, as a set of prognostic markers for performing an in vitro methods as disclosed above. In particular, the said kit comprises no more than 20, preferably no more than 12, preferably no more than 10, preferably no more than 9, 8, 7, 6, 5, 4, 3, 2, or 1 reagent(s) for determining the expression level of a gene that does not belong to one of the above described.

In a first embodiment, the kit of the present invention is used for performing an in vitro method for identifying a MCL subject with a poor outcome that may benefit from a targeted therapeutic treatment as disclosed above.

In another embodiment, the kit of the present invention is used for performing an in vitro method for monitoring the efficacy of a therapeutic treatment targeting iron metabolism in a subject having MCL and undergoing said treatment.

The kits for detection of poor outcome B-Cell lymphoma, in particular MCL patients or respectively for monitoring the efficacy of a targeted therapeutic treatment, may also comprises all reagents needed for the detection and/or quantification of expression of the said genes or proteins of interest according to the invention.

In a particular embodiment, the kit dedicated to MCL subjects comprises a set of probe sets for measuring the expression level of 8 genes and/or proteins encoded by the said 8 genes selected in the group consisting of APEX1, TFRC, HIF1A, ABCG2, SCARA3, IREB2, SFXN4 and SLC39A14. In particular, the said kit comprises no more than 20, preferably no more than 12, preferably no more than 10, preferably no more than 9, 8, 7, 6, 5, 4, 3, 2, or 1 reagent(s) for determining the expression level of a gene that does not belong to one of the above described. The kit may also comprise generic reagents useful for the determination of the expression level of any gene, such as Taq polymerase or an amplification buffer.

Pharmaceutical Composition

Another object of the invention is a pharmaceutical composition comprising, in a pharmaceutical acceptable vehicle, a molecule targeting iron metabolism, in particular an iron chelator or small molecule sequestering lysosomal iron, in particular selected in the group consisting of Deferasirox, Deferoxamine, Deferiprone, Salinomycin, analogs or derivatives thereof, preferably salinomycin and nitrogen-containing analogs of salinomycin, for use in a method for treating Mantle cell lymphoma (MCL) subjects.

In particular, the said pharmaceutical composition is used in a method for treating subjects identified according to the in vitro method of the invention as having a poor outcome according to iron-score and consequently likely to display a MCL relapse and/or death.

Examples of nitrogen-containing analogs of salinomycin are disclosed in the WO2016/038223.

In a particular embodiment, the iron chelator is a nitrogen-containing analog of salinomycin of formula (I)

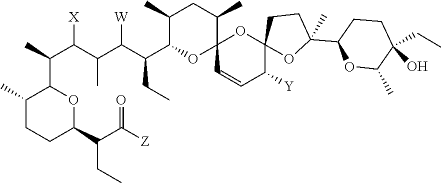

wherein:
—W is selected from the group consisting of ═O; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈;
—X is selected from the group consisting of ═O, —OH; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
—Y is selected from the group consisting of —OH; ═N—OH; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
   R₁ and R₂, identical or different, are selected from the group consisting of H; (C₁-C₁₆)-alkyl; (C₃-C₁₆)-alkenyl; (C₃-C₁₆)-alkynyl; (C₃-C₁₆)-cycloalkyl; aryl; heteroaryl; (C₁-C₆)-alkyl-aryl; (C₁-C₆)-alkyl-heteroaryl; or R₁ represents H and R₂ represents OR₉, where R₉ is H, (C₁-C₆)-alkyl, aryl and (C₁-C₆)-alkyl-aryl;
   R₃ is selected from the group consisting of H; (C₁-C₆)-alkyl; (C₁-C₆)-alkyl-aryl; R₄ and R₅, identical or different, are selected from the group consisting of H; (C₁-C₆)-alkyl; aryl and (C₁-C₆)-alkyl-aryl;
   R₆, R₇ and R₈, identical or different, are selected from the group consisting of (C₁-C₆)-alkyl; aryl and (C₁-C₆)-alkyl-aryl;
—Z is a group such as OH; NHNR₉R₁₀; NHOC(O)R₁₁; N(OH)—C(O)R₁₁; OOH, SR₁₂; 2-aminopyridine; 3-aminopyridine; —NR₃—(CH₂)ₙ—NR₄R₅; and —NR₃—(CH₂)ₙ—OH; where:
R₉ and R₁₀, identical or different, are selected from the group consisting of H, (C₁-C₆)-alkyl, aryl and (C₁-C₆)-alkyl-aryl;
R₁₁ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl;
R₁₂ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl n=0, 2, 3, 4, 5 or 6,
with the proviso that at least one of W, X and Y is selected from the group consisting of —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈.

Advantageously, R₁ and R₂, identical or different, are selected from the group consisting of H; (C₁-C₁₆)-alkyl, advantageously (C₃-C₁₄)-alkyl, more advantageously (C₈-C₁₄)-alkyl; (C₃-C₁₆)-alkenyl, advantageously (C₃-C₅)-alkenyl; (C₃-C₁₆)-alkynyl, advantageously (C₃-C₅)-alkynyl; (C₃-C₁₆)-cycloalkyl, advantageously (C₃-C₆)-cycloalkyl; (C₁-C₆)-alkyl-aryl, advantageously benzyl, and (C₁-C₆)-alkyl-heteroaryl, advantageously CH₂-pyridynyl.

Advantageously, R₁ and R₂ are not both H.

More advantageously, R₁ is H and R₂ is selected from the group consisting of (C₁-C₁₆)-alkyl, advantageously (C₃-C₁₄)-alkyl, more advantageously (C₈-C₁₄)-alkyl; (C₃-C₁₆)-alkenyl, advantageously (C₃-C₅)-alkenyl; (C₃-C₁₆)-alkynyl, advantageously (C₃-C₅)-alkynyl; (C₃-C₁₆)-cycloalkyl, advantageously (C₃-C₆)-cycloalkyl; (C₁-C₆)-alkyl-aryl, advantageously benzyl, and (C₁-C₆)-alkyl-heteroaryl, advantageously CH₂-pyridynyl.

Advantageously, R₃ is selected from the group consisting of H and (C₁-C₆)-alkyl. Preferably, R₃ is H.

Advantageously, Z is OH, OOH, NHNH₂, NHOH, or NH₂OH, preferably OH

In a preferred embodiment, the iron chelator is a compound of formula (I) as defined above, wherein X is OH, Z is OH and Y is NR₁R₂ where R₁ is H and R₂ is selected from the group consisting of (C₁-C₁₆)-alkyl, advantageously (C₈-C₁₄)-alkyl; (C₃-C₁₆)-alkenyl, advantageously (C₃-C₅)-alkenyl; (C₃-C₁₆)-alkynyl, advantageously (C₃-C₅)-alkynyl and (C₃-C₁₆)-cycloalkyl, advantageously (C₃-C₆)-cycloalkyl; (C₁-C₆)-alkyl-aryl, advantageously benzyl, and (C₁-C₆)-alkyl-heteroaryl, advantageously CH₂-pyridynyl.

In a more preferred embodiment, the iron chelator is a compound of formula (I) as defined above, wherein W is ═O, X is OH, Z is OH, and Y is NR₁R₂ where R₁ is H and R₂ is selected from the group consisting of (C₃-C₅)-alkynyl and (C₃-C₆)-cycloalkyl, preferably (C₃-C₅)-alkynyl.

The compound of formula (I) wherein W is ═O, X is OH, Z is OH, and Y is NR₁R₂ where R₁ is H and R₂ is a (C₃-C₅)-alkynyl group, preferably propargyl, is also named Ironomycin or compound AM5 as disclosed in the patent application WO2016/038223.

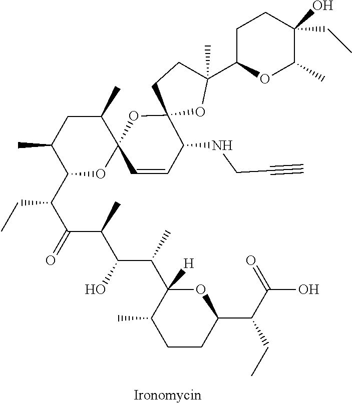 [SMILES: C#CCN[C@@H]1C=C[C@]2(O[C@H]([C@@H](CC)C(=O)[C@@H](C)[C@@H](O)[C@H](C)[C@@H]3O[C@@H]([C@@H](CC)C(=O)O)CC[C@@H]3C)[C@@H](C)C[C@H]2C)O[C@@]12CC[C@@](C)([C@H]1CC[C@](O)(CC)[C@H](C)O1)O2]

The compound of formula (I) wherein W is ═O, X is OH, Z is OH, and Y is NR₁R₂ where R₁ is H and R₂ is a (C₃-C₆)-cycloalkyl group, preferably cyclopropyl is also named AM23 as disclosed in the patent application WO2016/038223.

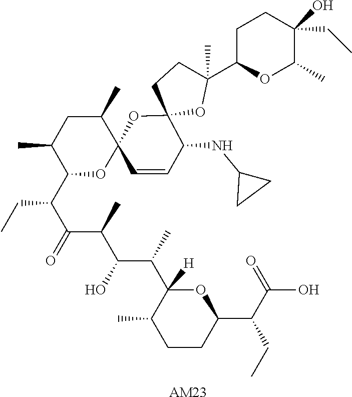

In another particular embodiment, W is ═O, X is OH, Z is OH, and Y is NR₁R₂ where R₁ is H and R₂ is a (C₃-C₆)-cycloalkyl group, in particular a substituted cyclopropyl as disclosed hereunder:

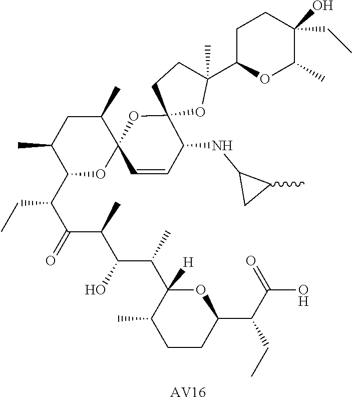 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](NC3CC3C)[C@]3(CC[C@@](C)([C@H]4CC[C@](O)(CC)[C@H](C)O4)O3)O2)[C@H](C)C[C@@H]1C ; "AV16"]

In another particular embodiment, W is ═O, X is OH, Z is OH, and Y is NR₁R₂ where R₁ is H and R₂ is a (C₁-C₆)-alkyl-aryl group, in particular a benzyl group substituted by an hydroxy, as disclosed hereunder:

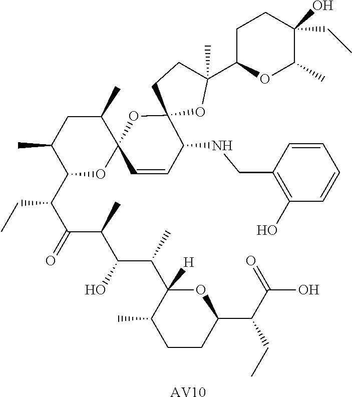 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](NCc3ccccc3O)[C@]3(CC[C@@](C)([C@H]4CC[C@](O)(CC)[C@H](C)O4)O3)O2)[C@H](C)C[C@@H]1C ; "AV10"]

In another particular embodiment, W is ═O, X is OH, Z is OH, and Y is NR₁R₂ where R₁ is H and R₂ is a (C₁-C₆)-alkyl-pyridyl group, in particular a CH₂-pyridinyl group, as disclosed hereunder:

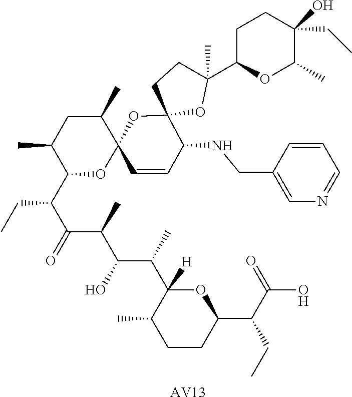 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](NCc3cccnc3)[C@]3(CC[C@@](C)([C@H]4CC[C@](O)(CC)[C@H](C)O4)O3)O2)[C@H](C)C[C@@H]1C ; "AV13"]

The compounds AM5, AM23, AV10, AV13 and AV16, preferably AM5 are particular and preferred compounds used in the pharmaceutical composition, pharmaceutical product and therapeutic uses disclosed hereunder.

The pharmaceutical composition for use according to the invention comprises at least one compound of formula (I) as defined above, a pharmaceutical salt, solvate or hydrate thereof, and at least one pharmaceutically acceptable excipient.

For the purpose of the invention, the term ‘pharmaceutically acceptable’ is intended to mean what is useful to the preparation of a pharmaceutical composition, and what is generally safe and non-toxic, for a pharmaceutical use.

The term «pharmaceutically acceptable salt, hydrate of solvate» is intended to mean, in the present invention, a salt of a compound which is pharmaceutically acceptable, as defined above, and which possesses the pharmacological activity of the corresponding compound.

Such salts comprise:
hydrates and solvates,
acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

The pharmaceutical compositions for use according to the invention can be intended to oral, sublingual, subcutaneous, intramuscular, intravenous, transdermal, topical or rectal administration. The active ingredient can be administered in unit forms for administration, mixed with conventional pharmaceutical carriers, to animals or to humans. When a solid composition is prepared in the form of tablets, the main active ingredient is mixed with a pharmaceutical vehicle and other conventional excipients known to those skilled in the art.

The compounds of the invention can be used in a pharmaceutical composition at a dose ranging from 0.01 mg to 1000 mg a day, administered in only one dose once a day or in several doses along the day, for example twice a day. The daily administered dose is advantageously comprised between 5 mg and 500 mg, and more advantageously between 10 mg and 200 mg. However, it can be necessary to use doses out of these ranges, which could be noticed by the person skilled in the art.

The invention also concerns a method for treating MCL subjects, preferably having a poor outcome as identified by the in vitro method of the invention, more preferably a MCL subject having a poor outcome as identified by the in vitro method of the invention, which method comprises (i) determining whether the subject is likely to have a relapse and/or death, by the in vitro method according to the invention and based on iron score, and (ii) administering a molecule targeting iron metabolism to said subject if the subject has been determined to have a ‘poor outcome’.

The method may further comprise, if the subject has been determined to be unlikely to have a ‘poor outcome’ a step (iii) of administering an alternative anticancer treatment to the subject Such alternative anticancer treatment depends on the specific B-Cell lymphoma and on previously tested treatments, but may notably be selected from radiotherapy, other chemotherapeutic molecules, or other biologics such as monoclonal antibodies directed to other antigens.

In certain embodiments, an anti-MCL treatment may include a treatment with anticancer compounds, radiation, surgery or stem cell transplant.

Pharmaceutical Product (Also Named “Combination Product”)

The present invention also relates to a pharmaceutical product comprising:
(i) a molecule targeting iron metabolism, in particular an iron chelator or a small molecule sequestering lysosomal iron and
(ii) another anti-cancer agent selected from the group consisting of agents used either in chemotherapy, in targeted treatments, in immune therapies, and in combinations thereof,

as combination product for simultaneous, separate or staggered use as a medicament in the treatment of MCL, in particular MCL subjects with a poor outcome according to in vitro method of the invention.

By ‘agents used in chemotherapy’ according to the invention, it means drugs also named ‘chemo drugs’ able to stop the growth of cancer cells, either by killing the cells or by stopping them from dividing.

By ‘agents used in targeted treatments’ according to the invention, it means drugs or other substances able to identify and attack specific types of cancer cells with less harm to normal cells. Some targeted therapies block the action of certain enzymes, proteins, or other molecules involved in the growth and spread of cancer cells. Other types of targeted therapies help the immune system kill cancer cells or deliver toxic substances directly to cancer cells and kill them. Targeted therapy may have fewer side effects than other types of cancer treatment. Most targeted therapies are either small molecule drugs or monoclonal antibodies.

By ‘agents used in immune therapies’ according to the invention, it means substances also named ‘immunomodulatory agents’ able to stimulate or suppress the immune system to help the body fight cancer. Some types of immunotherapy only target certain cells of the immune system. Others affect the immune system in a general way. Types of immunotherapy include as examples cytokines, and some monoclonal antibodies . . . .

In some embodiments, anticancer compounds may include a chemo drug, in particular selected in a group comprising vincristine, cyclophosphamide, etoposide, doxorubicin, liposomal doxorubicin, cytarabine, melphalan, Bendamustine, Cisplatin, daunorubicin, Fludarabine, Methotrexate.

In some embodiments, anticancer compounds may include:
Bcl2 inhibitors, or
BTK inhibitors,
and mixtures thereof.

‘Bcl-2 (B-cell lymphoma 2) inhibitors’ are a class of compounds that inhibit Bcl-2 family of regulator proteins that regulate cell death (apoptosis), by either inhibiting (anti-apoptotic) or inducing (pro-apoptotic) apoptosis. They are used to selectively induce apoptosis in malignant cells. Mention may be made of ABT-737 and navitoclax (ABT-263), and preferably Venetoclax (ABT-199, CAS No.: 1257044-40-8) that is a highly selective inhibitor, which inhibits Bcl-2, but not Bcl-xL or Bcl-w.

‘Bruton's tyrosine kinase’ (abbreviated Btk or BTK)’ inhibitors, also known as tyrosine-protein kinase BTK inhibitors, are a class of compounds that inhibit a tyrosine kinase that plays a crucial role in B cell development. Mention may be made of Ibrutinib (PCI-32765, CAS No. 936563-96-1).

In some embodiments, anticancer compounds may include a proteasome inhibitor, in particular selected in a group comprising bortezomib, carfilzomib and ixazomib.

In some embodiments, the immunomodulatory agent is selected in a group comprising thalidomide, lenalidomide, pomalidomide and a derivative thereof.

In some embodiments, anticancer compounds may include a corticosteroid, in particular selected in a group comprising dexamethasone and prednisone.

In some embodiments, anticancer compounds may include an epidrug including histone deacetylase (HDAC) inhibitor, DNMT inhibitor, EZH2 inhibitor, BET inhibitor, PRMT5 inhibitor, IDH inhibitor.

In some embodiments, anticancer compounds may include a monoclonal antibody, in particular selected in a group comprising Rituximab and obinutuzumab.

In some embodiments, anticancer compounds may include immunotherapy with CAR-T cells, in particular selected in a group comprising Tisagenlecleucel and axicabtagene ciloleucel and lisocabtagene maraleucel

In a particular embodiment, for treatment of MCL subjects, the molecule targeting iron metabolism, in particular an iron chelator or a small molecule sequestering lysosomal iron (i) is selected in the group consisting of Deferasirox, Deferoxamine, Deferiprone, Salinomycin, analogs or derivatives thereof, preferably salinomycin and nitrogen-containing analogs of salinomycin as defined above, and the other anti-cancer agent (ii) is selected from the group consisting of agents used in chemotherapy (iia), in particular cyclophosphamide, doxorubicin, or etoposide, Venetoclax, Ibrutinib, and combinations thereof.

In a particular and preferred embodiment for MCL treatment, the iron chelator (i) is a compound of formula (I) as defined above, wherein W is ═O, X is OH, Z is OH, and Y is NR₁R₂ where R₁ is H and R₂ is selected from the group consisting of (C₃-C₅)-alkynyl and (C₃-C₆)-cycloalkyl, preferably (C₃-C₅)-alkynyl and the other chemotherapy compound (ii) is Doxorubicin, Venetoclax, or Ibrutinib.

Another preferred subject-matter of the invention for MCL treatment is a pharmaceutical product or composition comprising:
(i) a compound of formula (I) as defined above, wherein W is ═O, X is OH, Z is OH, and Y is NR₁R₂ where R₁ is H and R₂ is selected from the group consisting of (C₃-C₅)-alkynyl and (C₃-C₆)-cycloalkyl, preferably (C₃-C₅)-alkynyl and
(ii) a chemotherapy compound selected in the group consisting of cyclophosphamide, doxorubicin, etoposide, Venetoclax, or Ibrutinib preferably doxorubicin, Venetoclax, or Ibrutinib.

The present invention will be now illustrated by the non-limitative examples.

### Examples

Material and Methods

Gene Expression Data Analyses and Building of the Iron Score

The list of 62 genes involved in the regulation of iron biology was established using previously published data (Miller et al., 2011).

Expression of these genes was interrogated in ML samples (n=71). Affymetrix gene expression data are publicly available via the online Gene Expression Omnibus (http://www.ncbi.nm.nih.gov/geo/) under accession number GSE0793.

The related 63 genes involved in the iron metabolism are listed in the table 3 hereunder:

TABLE 3

| Gene Symbol | Gene Title |
|---|---|
| FTH1 | ferritin, heavy polypeptide 1 |
| EPAS1 | endothelial PAS domain protein 1 |
| HIF1A | hypoxia inducible factor 1, alpha subunit (basic helix-loop-helix |
| | transcription factor) |
| LTF | lactotransferrin |
| UROS | uroporphyrinogen III synthase |
| HMBS | hydroxymethylbilane synthase |
| FECH | ferrochelatase |
| SLC11A2 | solute carrier family 11 (proton-coupled divalent metal ion |
| | transporter), member 2 |
| ABCB6 | ATP binding cassette subfamily B member 6 (Langereis blood group) |
| HMOX1 | heme oxygenase 1 |
| HEPH | hephaestin |
| PPOX | protoporphyrinogen oxidase |
| CP | ceruloplasmin (ferroxidase) |
| MTF1 | metal-regulatory transcription factor 1 |
| STEAP1 | six transmembrane epithelial antigen of the prostate 1 |
| FXN | frataxin |
| ALAS1 | 5′-aminolevulinate synthase 1 |
| LRP2 | LDL receptor related protein 2 |
| ACO1 | aconitase 1, soluble |
| HP///HPR | haptoglobin///haptoglobin-related protein |
| TFRC | transferrin receptor |
| UROD | uroporphyrinogen decarboxylase |
| FBXL5 | F-box and leucine-rich repeat protein 5 |
| ISCU | iron-sulfur cluster assembly enzyme |
| ISCA1 | iron-sulfur cluster assembly 1 |
| ABCG2 | ATP binding cassette subfamily G member 2 (Junior blood group) |
| TFR2 | transferrin receptor 2 |
| HFE | hemochromatosis |
| SLC39A14 | solute carrier family 39 (zinc transporter), member 14 |
| LCN2 | lipocalin 2 |
| FTL | ferritin, light polypeptide |
| TMPRSS6 | transmembrane protease, serine 6 |
| CIAO1 | cytosolic iron-sulfur assembly component 1 |
| HMOX2 | heme oxygenase 2 |
| STEAP3 | STEAP family member 3, metalloreductase |
| NFS1 | NFS1 cysteine desulfurase |
| ALAD | aminolevulinate dehydratase |
| SLC22A17 | solute carrier family 22, member 17 |
| NARFL | nuclear prelamin A recognition factor-like |
| NFU1 | NFU1 iron-sulfur cluster scaffold |
| HAMP | hepcidin antimicrobial peptide |
| SFXN3 | sideroflexin 3 |
| CYBRD1 | cytochrome b reductase 1 |
| SLC25A37 | solute carrier family 25 (mitochondrial iron transporter), member 37 |
| FLVCR1 | feline leukemia virus subgroup C cellular receptor 1 |
| SLC40A1 | solute carrier family 40 (iron-regulated transporter), member 1 |
| SLC25A28 | solute carrier family 25 (mitochondrial iron transporter), member 28 |
| MON1A | MON1 secretory trafficking family member A |
| SFXN4 | sideroflexin 4 |
| STEAP2 | STEAP family member 2, metalloreductase |
| IREB2 | iron responsive element binding protein 2 |
| STEAP4 | STEAP family member 4 |
| ISCA2 | iron-sulfur cluster assembly 2 |
| SFXN2 | sideroflexin 2 |
| HFE2 | hemochromatosis type 2 (juvenile) |
| SFXN1 | sideroflexin 1 |
| SFXN5 | sideroflexin 5 |
| SCARA5 | scavenger receptor class A, member 5 |
| APEX2 | APEX nuclease (apurinic/apyrimidinic endonuclease) 2 |
| HPX | hemopexin |
| HIF1AN | hypoxia inducible factor 1, alpha subunit inhibitor |
| SCARA3 | Scavenger Receptor Class A Member 3 |

Significance analysis of microarray analysis was applied to the 62 selected probe sets in the different samples with 1000 permutations, a fold change of two and a false discovery rate of 0% (t-test).

Gene expression microarray data from a cohort of 71 patients with MCL was used. Affymetrix gene expression data are publicly available via the online Gene Expression Omnibus (http://www.ncbi.nim.nih.gov/geo/) under accession number GSE10793. They were performed using Lymphochip cDNA microarray for the cohort of 71 patients. The data were analyzed with Microarray Suite version 5.0 (MAS 5.0), using Affymetrix default analysis settings and global scaling as normalization method. The trimmed mean target intensity of each array was arbitrarily set to 500.

In the cohort, the statistical significance of overall survival (OS) of the expression of each probe set of the iron list was calculated by the log-rank test. Multivariate analysis was performed using the Cox proportional hazards model. Survival curves were plotted using the Kaplan-Meier method in the platform Genomicscape (Kassambara et al., 2015). Probe sets with a common prognosis value in the cohort were selected. To gather their prognostic information within one parameter, the Iron Score of MCL was built as the sum of the beta coefficients weighted by ±1 according to the patient signal above or below the probe set Maxstat value (Kassambara et al., 2012).

Human MCL Cell Lines

The 6 MCL cell lines (GRANTA-519, JEKO-1, MINO, MAVER-1, JVM-2 and REC-1) were purchased from the DSMZ (Leibniz-Institut DSMZ—Deutsche Sammlung von Mikroorganismen und Zellkulturen GmbH, Germany). They were maintained according to the supplier's recommendations. Cultures were maintained at 37° C. in a humidified atmosphere with 5% CO₂.

Reagents

Deferoxamine (from Novartis Pharma SAS) was dissolved in sterile distilled water and Deferasirox (from Selleckchem S1712), was dissolved in dimethyl sulfoxide (DMSO) to a concentration of 300 mM and 50 mM respectively. Ironomycin also named ‘AM5’ in the patent application WO2016/038223 was dissolved in dimethyl sulfoxide (DMSO) to a concentration of 10 mM. Doxorubicin (From Sellekchem S1208, 20 mM in DMSO), Venetoclax (from Sellekchem, S8048 10 mM in DMSO), Ibrutinib (From Sellekchem S2680, 50 mM in DMSO).

Cell Viability Assay

MCL cell lines were cultured for 4 days in 96-well flat-bottom microtiter plates in RPMI 1640 medium or DMEM medium, 10% or 20% FCS (control medium) in the presence of various compounds. The number of viable cells in culture was determined using the CellTiter-Glo Luminescent Cell Viability Assay from Promega, Madison, Wis., USA using a Centro LB 960 luminometer (Berthold Technologies, Bad Wildbad, Germany).

This test is based on quantitation of the intracellular ATP present, which signals the presence of metabolically active cells. Data are expressed as the mean percentage of six replicates, normalized to the untreated control.

Flow Cytometry Analyses

AnnexinV-PE staining for apoptosis analysis was performed using the “PE Annexin V Apoptosis Detection Kit I” (559763, Becton Dickinson).

The cell cycle progression was studied by flow cytometry using the Apoptosis, DNA Damage, and Cell Proliferation Kit (562253, Becton Dickinson). Briefly, cells were labeled with bromodeoxyuridine (BrdU), an analog of the DNA precursor thymidine that can be incorporated into newly synthesized DNA and detected with an antibody against BrdU to measure cell proliferation. After this labeling, the cells were fixed, permeabilized and treated with DNase to expose the BrdU epitopes. Following this treatment, cells were simultaneously stained with fluorochrome-labeled anti-BrdU, anti-cleaved Poly ADP-ribose polymerase 1 (PARP), anti-H2AX phosphorylated at serine 139. They were also stained with DAPI to determine DNA content. Finally, cells were resuspended in staining buffer and analyzed by flow cytometry (Fortessa, Becton Dickinson).

Primary MCL Cells

Lymph node samples were collected after patients' written informed consent in accordance with the Declaration of Helsinki and institutional research board approval from Montpellier University hospital. Cells are obtained from lymph nodes or blood of 9 patients with MCL. Cells from blood or bone marrow are obtained by density gradient separation and cells from lymph node are obtained with a tissues dissociator and qualified by Flow cytometry.

Cells are cultured in Gibco® Iscove's MDM (Glutamax) medium (#31980-022) with 20% FBS with antibiotitcs-antimicotics (Gibco Penicillin-streptomycin-amphotericin B 100X, #15240-096) at a density of 0.5×10⁶ Cell/mL with 50 ng/mL of histidine-tagged CD40L (R&D System, 2706-CL) and 5 μg/mL of anti-histidine antibody R&D System, MAB050), Gibco @pyruvate 100X, #1136-039. Cells are seeded 24H after thawing and treated with various compounds during 72H.

Total cells were counted with trypan bleu and stained with the panel CD45 V500 (BD, #560777), Kappa FITC (Dako, F0434), CD19 PE-Cy7 (BD, #341113), Lambda PE (Dako, R0437), CD3 APC-H7 (BD, #641415), CD10 APC (BD, #332777) and CD20 V450 (BD, #655872) and analyzed by flow cytometry (Canto II cytometer, BD Pharmigen). Tumorous MCL cells were gated on CD19+, CD45+, CD20+, Kappa or lambda.

Western-Blot:

The total cell lysates were obtained with RIPA 1× lysis buffer (#9806, Cell Signaling®) according with the supplier recommendations.

Protein lysates migrate on 10% polyacrylamide gel (NP-0301, Novex, Life Technologies®), in MOPS 1× running buffer (NP0001, Novex, Life Technologies®) or MES 1× running buffer NP-0002, Novex, Life Technologies®) and proteins are transferred to nitrocellulose membrane (1B301001, I-Blot Transfert Starck, NuPAGE, Life Technologies®). The primary antibodies mouse-anti-phospho-Histone H2A.X (Ser139) clone JBW301 (1/1000, Merck Millipore), rabbit anti-cyclinD1 (#2926, 1/1000, Cell Signaling®), rabbit anti-phospho (S795)-Rb (#9301, 1/1000, Cell Signaling®), mouse anti-Rb (#9309, 1/1000, Cell Signaling®), mouse anti-CDK4 (#2906, 1/1000, Cell Signaling®) were incubated in TBS-Tween 20 0.1% (Tris-Buffered Saline, pH 7.4) with 5% non-fat milk or Bovine serum albumin (Sigma-Aldrich, A7906). Protein levels are objectified by labeling with an anti-β-actin mouse monoclonal antibody (Sigma, A5441, St Louis, Mo., USA 1/1000). Primary antibodies are visualized with secondary anti-rabbit antibodies (Sigma®, A9169) or anti-mouse antibodies (Jackson, 115-036-068) coupled to peroxidase allows the development by chemiluminescence by Western Lightning ECL (NEL121001EA, Perkin Elmer®). Quantification of protein levels was performed with Image J® software (National Institutes of Health, Bethesda, Md., USA).

Quantification of the Interaction Effect

The interaction between the drugs tested in vitro was investigated with a concentration matrix test, in which increasing concentration of each single drug were assessed with all possible combinations of the other drugs. For each combination, the percentage of expected growing cells in the case of effect independence was calculated according to the Bliss equation (Combes et al., 2019):

fuC=fuA·fuB

where fuC is the expected fraction of cells unaffected by the drug combination in the case of effect independence, and fuA and fuB are the fractions of cells unaffected by treatment A and B, respectively. The difference between the fraction of living cells in the cytotoxicity test and the fuC value was considered as an estimation of the interaction effect, with positive values indicating synergism and negative values antagonism.

Synergy matrix was built with the R package “SynergyFinder”.

Results:

Considering the important role of iron metabolism in cancer cell biology, the inventors first aimed to identify iron metabolism genes associated with a prognostic value in MCL. A list of 63 genes involved in the regulation of iron biology was extracted from literature (Miller et al., 2011), as disclosed in the materiel and methods above (table 3).

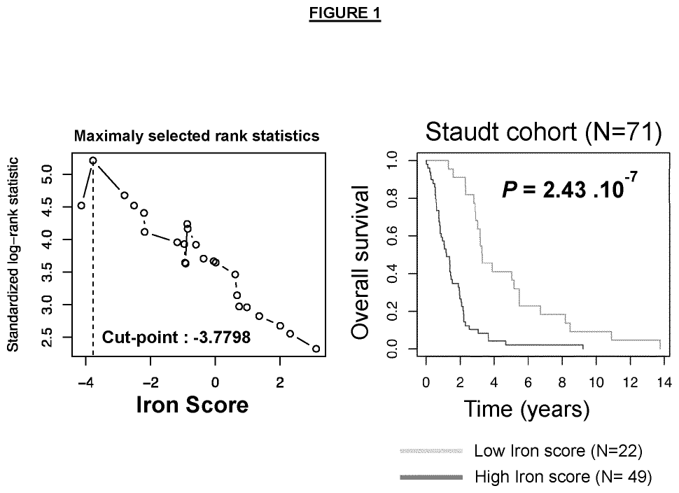

Using Maxstat R function and Benjamini Hochberg multiple testing correction (Lausen and Schumacher, 1992), 8 genes demonstrated a prognostic value in a cohort of MCL patients (n=71) (FIG. 1), as disclosed above and reported in the table 4 hereunder:

TABLE 4
Probsets and names of prognostic genes of the Iron score

| Probe set | | |
|---|---|---|
| Lymphochip cDNA | | |
| microarray | Gene | Gene name |
| 17615_1 | APEX1 | DNA-(apurinic or apyrimidinic site) |
| | | lyase |
| 17881_1 | TFRC | Transferrin Receptor Protein 1 |
| 26166_1 | ABCG2 | ATP-binding cassette transporter |
| | | G2 |
| 29606_1 | SCARA3 | Scavenger Receptor Class A |
| | | Member 3 |
| 29976_1 | IREB2 | Iron Responsive Element Binding |
| | | Protein 2 |
| 32684_1 | SLC39A14 | Solute Carrier Family 39 Member |
| | | 14 |
| 32719_1 | SFXN4 | sideroflexin 4 |
| 33901_1 | HIF1A | Hypoxia inductible factor A 1 |

As illustrated in the table 2 below and the table 5 hereunder with hazard ratio (HR), high expression of four genes was associated with a good prognosis (‘good outcome’) including ABCG2 (ATP-binding cassette transporter G2), SCARA3 (Scavenger Receptor Class A Member 3), IREB2 (Iron Responsive Element Binding Protein 2) and SFXN4 (sideroflexin 4); and high expression of four genes was associated with a poor prognosis (‘poor outcome’): APEX1 (DNA-(apurinic or apyrimidinic site) lyase), TFRC (Transferrin Receptor Protein 1), SLC39A14 (Solute Carrier Family 39 Member 14), and HIF1A (Hypoxia inductible factor A 1).

TABLE 5

| Probeset | Gene | HR | Prognostic |
|---|---|---|---|
| 17615_1 | APEX1 | 2.19804037 | Bad |
| 17881_1 | TFRC | 1.88605426 | Bad |
| 26166_1 | ABCG2 | 0.3289553 | Good |
| 29606_1 | SCARA3 | 0.51308035 | Good |
| 29976_1 | IREB2 | 0.45059709 | Good |
| 32684_1 | SLC39A14 | 4.90822285 | Bad |
| 32719_1 | SFXN4 | 0.29250155 | Good |
| 33901_1 | HIF1A | 2.25953813 | Bad |

We next combined the prognostic information of these genes in a GEP-based iron-score. The iron score is defined by the sum of the beta coefficients of the Cox model for each prognostic gene, weighted by −1 according to the patient MMC signal above or below the probe set Maxstat value as previously described (Herviou et al., 2018). Maxstat algorithm segregated the Staudt cohort into two groups with 69% of the patients with an iron score >−3.7798 and 31% of the patients with an iron score ≤−3.7798 with a maximum difference in overall survival (OS; FIG. 1). Patients with high-risk iron score have a median OS of 1.1 year versus 3.3 years for patients with low iron score (P=2.43·10⁻⁷) in the Staudt cohort (FIG. 1).

These data demonstrated that high iron score allows to identify MCL patient with a poor outcome and dysregulation of iron metabolism that could benefit from targeted therapy.

Ironomycin Kills MCL Cells with Nanomolar Concentration

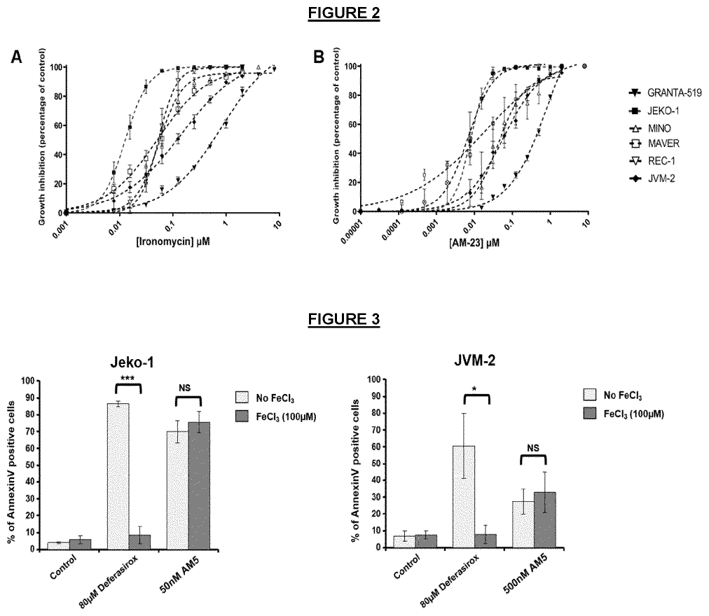

We investigated the therapeutic interest of iron metabolism inhibitors AM5 (Ironomycin) and AM23. The panel of 6 MCL cell lines were incubated with increasing concentrations of Ironomycin (FIG. 2 A), AM23 (FIG. 2 B) or vehicle for 96H (A). Inhibitory concentration 50% (IC₅₀) was calculated with concentration-response curve after treatment with Ironomycin. Cell viability was examined using quantification of ATP assay. Data are expressed as mean percentage+/−SEM of at least three independent experiments performed in sixplicate. Tables 6 and 7 presenting the inhibitory concentration 50% (IC₅₀) of Ironomycin and AM23 for the 6 MCL cell-lines.

TABLE 6

| MCL cell lines | IC₅₀ Ironomycin (nM) |
|---|---|
| GRANTA-519 | 802 |
| JEKO-1 | 12.8 |
| MINO | 54.4 |
| MAVER-1 | 50.5 |
| REC-1 | 53.4 |
| JVM-2 | 139.4 |

TABLE 7

| MCL | IC₅₀ AM-23 (nM) |
|---|---|
| GRANTA-519 | 845 |
| JEKO-1 | 8 |
| MINO | 43 |
| MAVER-1 | 17.3 |
| REC-1 | 6.8 |
| JVM-2 | 58 |

Targeting Iron Metabolism Induces MCL Cell Toxicity

The inventors further investigated the effect of Iron supplementation on the cell death induced by these treatments. Iron chelator (Deferasirox) concentrations were chosen according the maximal plasmatic concentration achievable in the patient (Nisbet-Brown et al., 2003). The inventors demonstrated that Iron chelators and Ironomycin induces apoptosis in Jeko-1 and JVM-2 MCL cell lines monitored by Annexin V staining. Iron supplementation significantly inhibited the effect of iron chelator on MCL cells apoptosis (P<0.01 for Deferasirox treatment). However, iron supplementation did not affect ironomycin-induced MCL cell cytotoxicity. So interestingly, when compared to Deferasirox (iron chelator), the toxicity mediated by ironomycin in Jeko-1 and JVM-2 MCL cell lines could not be reversed by iron supplementation (FIG. 3).

Ironomycin Affects MCL Cell Division and Induces DNA Damage Response.

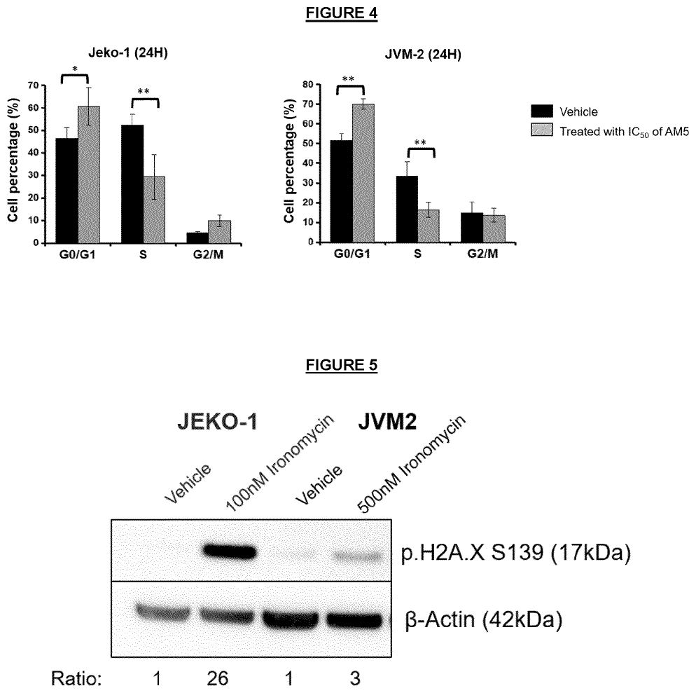

Cells were incubated with vehicle or with IC₅₀ Ironomycin for 24 hours. Cell cycle was analyzed using flow cytometry, S phase was stained by an anti-BrdU antibody after BrdU incorporation and DNA content was strained by 4′,6-diamidino-2-phenylindole (DAPI) for Jeko-1 and JVM-2 cell lines. Histograms represent the mean percentage and SD of each cell cycle phase of three independent experiments. * and ** indicate a significant difference of P<0.05 and P<0.01, respectively with paired student t-test. (FIG. 4)

Ironomycin induces DNA damage response: double strand breaks evidenced by Serine 139 phosphorylation of histone variant H2A.X. Cells were treated with Ironomycin (100 nM for Jeko-1 and 500 nM for JVM-2) during 24H. Protein levels of Phospho-H2A.X (S139) were analyzed by western blot and normalized by β-actin protein level (FIG. 5).

Ironomycin Induces a Significant Downregulation of Cyclin D1 in MCL Cell Lines

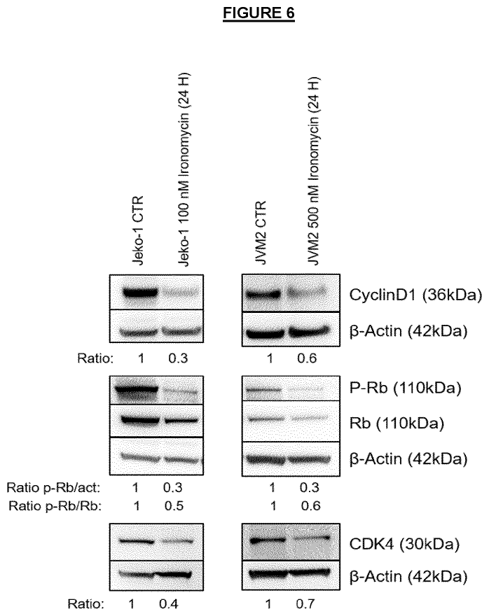

Mantle cell lymphoma (MCL) is now recognized as an aggressive B-cell lymphoma with various growth patterns (mantle zone, nodular, or diffuse) and a broad range of cytologic features. Most cases of MCL exhibit a characteristic phenotype (CD20*, CD5*, CD43*, CD3-, CD10-, CD23-) and have the t(11;14)(q13;q32) with overexpression of the cyclin D1 (CCND1) gene on chromosome 11q13 (Banks et al., 1992). Cyclin D1, a D-type cyclin that is not expressed in normal B lymphocytes, plays a key role in cell cycle regulation during the G₁ to S phase transition by binding to cyclin-dependent kinase 4 (CDK4) and CDK6, resulting in phosphorylation and inactivation of the retinoblastoma protein (RB)(Matsushime et al., 1994; Meyerson et al, 1994; Mittnacht et al., 1994). Of major interest, we identified that ironomycin induces a significant downregulation of Cyclin D1 in MCL cell lines. This Cyclin D1 downregulation is associated with a downregulation of RB phosphorylation and CDK4 protein levels (FIG. 6).

Assays on Primary MCL Cells of Patients: Ironomycin Induces Cell Death

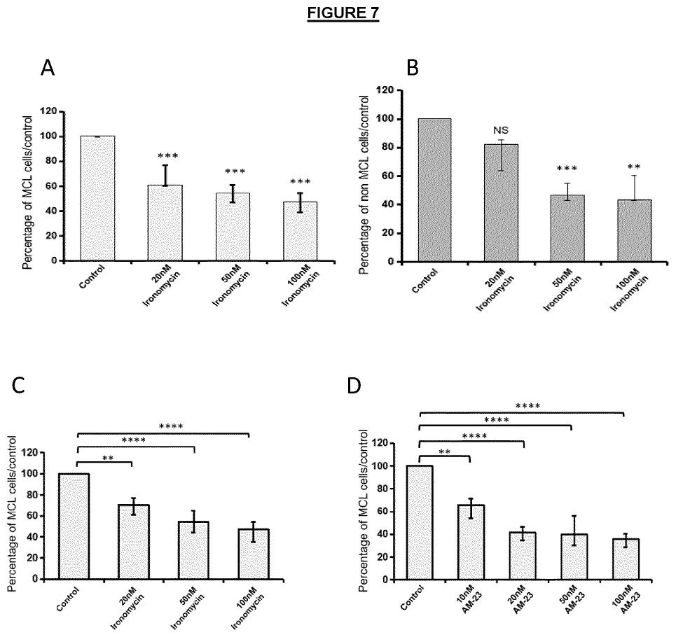

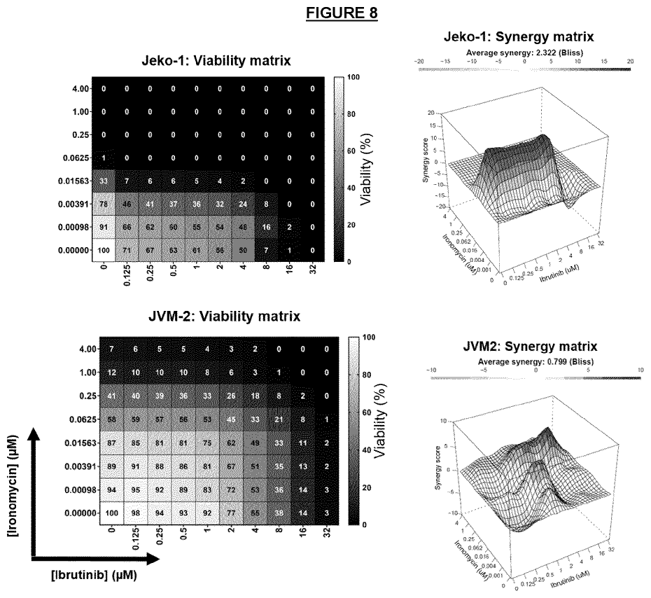

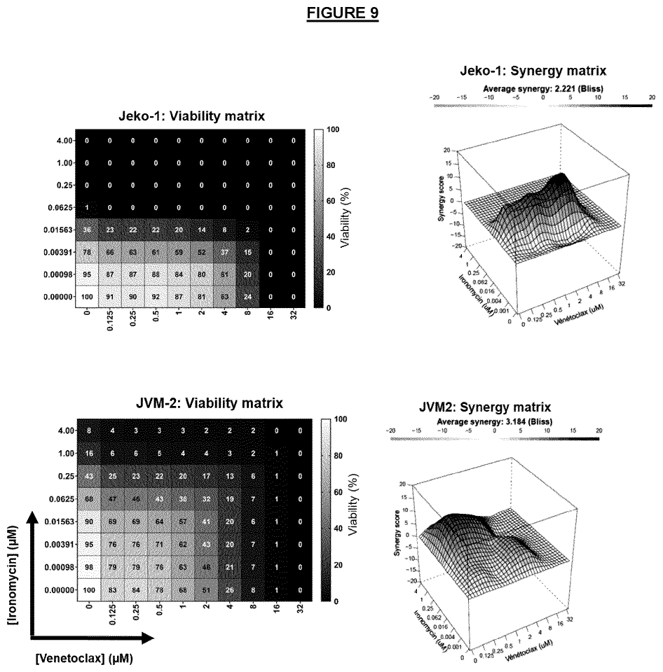

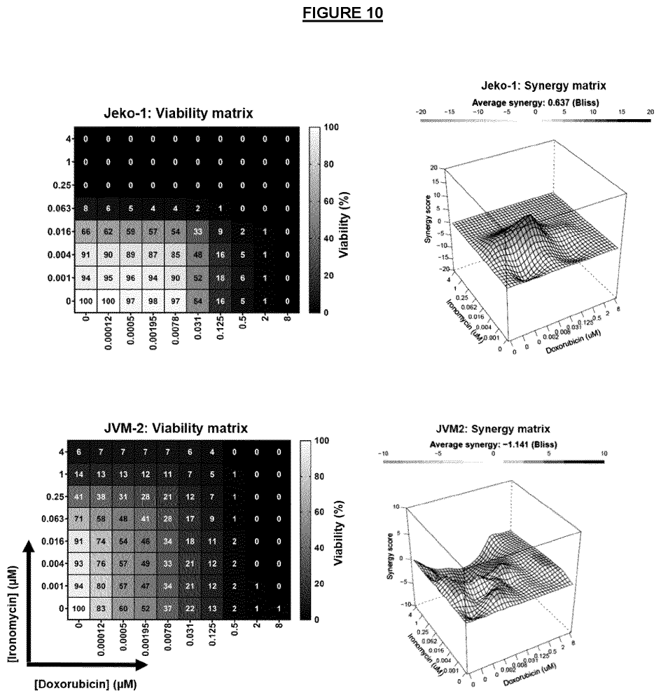

In a first experiment, primary MCL cells were treated with Ironomycin and incubated during 96H with CD40L. The toxicity on MCL cells (FIG. 7 A) and non-MCL cells (FIG. 7 B) was analyzed by flow cytometry and expressed in % of control.

To confirm that iron deprivation is of therapeutic interest in MCL, primary samples of MCL patients were cultured with their microenvironment recombinant CD40L in presence or absence of 20 nM, 50 nM and 100 nM of ironomycin. Ironomycin treatment significantly reduced the median number of viable primary MCL cells at 20 nM, 50 nM and 100 nM respectively (FIG. 7A). Interestingly, ironomycin (20 nM) demonstrated a higher toxicity in MCL cells compared to normal cells from the microenvironment (FIG. 7B).

These first results were further completed by additional experiments: primary samples of MCL patients were cultured with their microenvironment recombinant CD40L in presence or absence of 20 nM, 50 nM and 100 nM of ironomycin (FIG. 7 C) or 10 nM, 20 nM, 50 nM and 100 nM of AM-23 (FIG. 7 D). The toxicity on MCL cells was analyzed by flow cytometry and expressed in % of control.

Ironomycin treatment significantly reduced the median number of viable primary MCL cells (N=9) by 30% (P<0.01), 46% (P<0.0001) and 53% (P<0.0001), at 20 nM, 50 nM and 100 nM, respectively (FIG. 7C).

AM-23 treatment significantly reduced the median number of viable primary MCL cells (N=6) by 34% (P<0.01), 58% (P<0.0001), 60% (P<0.0001) and 64% (P<0.0001), at 10 nM, 20 nM, 50 nM and 100 nM, respectively (FIG. 7D).

Ironomycin Treatment Potentiates Conventional MCL Treatments.

We tested the therapeutic interest to combine Ironomycin with conventional chemotherapy used in MCL. Interestingly, we identified a synergistic effect when Ironomycin is combined with Ibrutinib BTK inhibitor (FIG. 8). Furthermore, we identified a synergistic effect when ironomycin is combined with Venetoclax Bcl2 inhibitor (FIG. 9) or with Doxorubicin (FIG. 10). These underlined a therapeutic interest to combine ironomycin with conventional drugs used in the treatment of MCL.

Altogether, these data underline that MCL patients my benefit from targeting iron homeostasis using Ironomycin or AM23 alone or in combination with conventional MCL treatments.

### REFERENCES

Banks P M, Chan J, Cleary M L, et al. Mantle cell lymphoma. A proposal for unification of morphologic, immunologic, and molecular data. Am J Surg Pathol. 1992; 16(7):637-640
Combes, E., Andrade, A. F., Tosi, D., Michaud, H.-A., Coquel, F., Garambois, V., Desigaud, D., Jarlier, M., Coquelle, A., Pasero, P., et al. Inhibition of Ataxia-Telangiectasia Mutated and RAD3-related (ATR) overcomes oxaliplatin resistance and promotes anti-tumor immunity in colorectal cancer. Cancer Res, 2019.
Herviou, L., Kassambara, A., Boireau, S., Robert, N., Requirand, G., Müller-Tidow, C., Vincent, L., Seckinger, A., Goldschmidt, H., Cartron, G., et al. (2018). PRC2 targeting is a therapeutic strategy for EZ score defined high-risk multiple myeloma patients and overcome resistance to IMiDs. Clin. Epigenetics 10, 121.
Kassambara, A., Hose, D., Moreaux, J., Walker, B. A., Protopopov, A., Reme, T., Pellestor, F., Pantesco, V., Jauch, A., Morgan, G., et al. (2012). Genes with a spike expression are clustered in chromosome (sub)bands and spike (sub)bands have a powerful prognostic value in patients with multiple myeloma. Haematologica 97, 622-630.
Kassambara, A., Réme, T., Jourdan, M., Fest, T., Hose, D., Tarte, K., and Klein, B. (2015). GenomicScape: an easy-to-use web tool for gene expression data analysis. Application to investigate the molecular events in the differentiation of B cells into plasma cells. PLoS Comput. Biol. 11, e1004077.
Lausen, B., and Schumacher, M. (1992). Maximally Selected Rank Statistics. Biometrics 48, 73-85.
Matsushime H, Quelle D E, Shurtleff S A, Shibuya M, Sherr C J, Kato J Y. D-type cyclin-dependent kinase activity in mammalian cells. Mol Cell Biol. 1994; 14(3):2066-2076.
Meyerson M, Harlow E. Identification of G1 kinase activity for cdk6, a novel cyclin D partner. Mol Cell Biol. 1994; 14(3):2077-2086.
Miller, L. D., Coffman, L. G., Chou, J. W., Black, M. A., Bergh, J., D'Agostino, R., Torti, S. V., and Torti, F. M. (2011). An iron regulatory gene signature predicts outcome in breast cancer. Cancer Res. 71, 6728-6737.
Mittnacht S, Lees J A, Desai D, Harlow E, Morgan D O, Weinberg R A. Distinct sub-populations of the retinoblastoma protein show a distinct pattern of phosphorylation. EMBO J. 1994; 13(1):118-127Moreaux J, Reme T, Leonard W, et al. Development of gene expression-based score to predict sensitivity of multiple myeloma cells to DNA methylation inhibitors. Mol Cancer Ther. 2012; 11(12):2685-2692.
Moreaux J, Reme T, Leonard W, et al. Gene expression-based prediction of myeloma cell sensitivity to histone deacetylase inhibitors. Br J Cancer. 2013; 109(3):676-685.
Nisbet-Brown, E., Olivieri, N. F., Giardina, P. J., Grady, R. W., Neufeld, E. J., Sechaud, R., Krebs-Brown, A. J., Anderson, J. R., Alberti, D., Sizer, K. C., et al. (2003). Effectiveness and safety of ICL670 in iron-loaded patients with thalassaemia: a randomised, double-blind, placebo-controlled, dose-escalation trial. Lancet Lond. Engl. 361, 1597-1602.

1.-13. (canceled)

14. An in vitro method for identifying a mantle cell lymphoma (MCL) subject with a poor outcome that may benefit from a therapeutic treatment targeting iron metabolism, comprising the steps of:
a) measuring the expression level of at least 1 gene and/or protein encoded by the said at least 1 gene selected from the group consisting of APEX1, TFRC, HIF1A, ABCG2, SCARA3, IREB2, SFXN4 and SLC39A14 involved in the iron metabolism, in a biological sample obtained from said subject;
b) calculating a score value from said expression level obtained at step a); and
c) classifying and identifying said subject as having a poor outcome according to the score value in comparison to a predetermined reference value (PRV).

15. The in vitro method according to claim 14, wherein the therapeutic treatment targeting iron metabolism is selected from the group consisting of iron chelators and small molecules sequestering lysosomal iron.

16. A kit dedicated to an in vitro method according to claim 14, comprising reagents for determining the expression level of at least 1 gene and/or protein selected from the group consisting of APEX1, TFRC, HIF1A, ABCG2, SCARA3, IREB2, SFXN4 and SLC39A14 in a sample of said subject.

17. The kit according to claim 16 dedicated to Diffuse larage B-cell lymphoma (DLBCL) subjects comprising a set of primers and/or probes for measuring the expression level of at least 3 genes and/or proteins encoded by the said at least 3 genes selected from the group consisting of APEX1, TFRC, HIF1A, ABCG2, SCARA3, IREB2, SFXN4 and SLC39A14.

18. A method for treating a subject having Mantle cell lymphoma (MCL) comprising administration to said subject a pharmaceutical composition comprising, in a pharmaceutical acceptable vehicle, an iron chelator or a small molecule sequestering lysosomal iron.

19. The method of claim 18, wherein said subject is identified as having a poor outcome that may benefit from a therapeutic treatment targeting iron metabolism according to the subject's iron score and consequently likely to have a relapse of MCL and/or death, wherein said identification is made via an in vitro method comprising the steps of:
a) measuring the expression level of at least 1 gene and/or protein encoded by the said at least 1 gene selected from the group consisting of APEX1, TFRC, HIF1A, ABCG2, SCARA3, IREB2, SFXN4 and SLC39A14 involved in the iron metabolism, in a biological sample obtained from said subject;
b) calculating a score value from said expression level obtained at step a); and
c) classifying and identifying said subject as having a poor outcome according to the score value in comparison to a predetermined reference value (PRV).

20. The method of claim 18, wherein the iron chelator present in the pharmaceutical composition is a nitrogen-containing analog of salinomycin of formula (I) 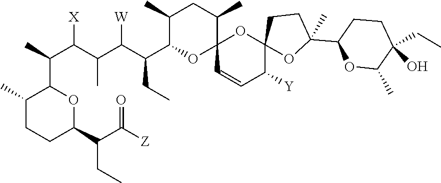
wherein:
—W is selected from the group consisting of ═O; —NR₁R₂; —NR₃—(CH₂)ₙ—NR₄R₅;
—O—(CH₂)ₙ—NR₄R₅; —NR₃—(CH₂)ₙ—N⁺R₆R₇R₈ and —O—(CH₂)ₙ—N⁺R₆R₇R₈;
—X is selected from the group consisting of ═O, —OH; —NR₁R₂; —NR₃—(CH₂)ₙ—NR₄R₅;
—O—(CH₂)ₙ—NR₄R₅; —NR₃—(CH₂)ₙ—N⁺R₆R₇R₈ and —O—(CH₂)ₙ—N⁺R₆R₇R₈,
—Y is selected from the group consisting of —OH; ═N—OH; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
R₁ and R₂, identical or different, are selected from the group consisting of H; (C₁-C₁₆)-alkyl; (C₃-C₁₆)-alkenyl; (C₃-C₁₆)-alkynyl; (C₃-C₁₆)-cycloalkyl; aryl; heteroaryl; (C₁-C₆)-alkyl-aryl; (C₁-C₆)-alkyl-heteroaryl; or R₁ represents H and R₂ represents OR₉, where R₉ is H, (C₁-C₆)-alkyl, aryl and (C₁-C₆)-alkyl-aryl;
R₃ is selected from the group consisting of H; (C₁-C₆)-alkyl; (C₁-C₆)-alkyl-aryl;
R₄ and R₅, identical or different, are selected from the group consisting of H; (C₁-C₆)-alkyl; aryl and (C₁-C₆)-alkyl-aryl;
R₆, R₇ and R₈, identical or different, are selected from the group consisting of (C₁-C₆)-alkyl; aryl and (C₁-C₆)-alkyl-aryl;
—Z is a group such as OH; NHNR₉R₁₀; NHOC(O)R₁₁; N(OH)—C(O)R₁₁; OOH, SR₁₂; 2-aminopyridine; 3-aminopyridine; —NR₃—(CH₂)ₙ—NR₄R₅; and —NR₃—(CH₂)ₙ—OH; where:
R₉ and R₁₀, identical or different, are selected from the group consisting of H, (C₁-C₆)-alkyl, aryl and (C₁-C₆)-alkyl-aryl;
R₁₁ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl;
R₁₂ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl n=0, 2, 3, 4, 5 or 6,
with the proviso that at least one of W, X, and Y is selected from the group consisting of —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈.

21. The method of claim 20, wherein the iron chelator present in the pharmaceutical composition is a nitrogen-containing analog of salinomycin of formula (I) 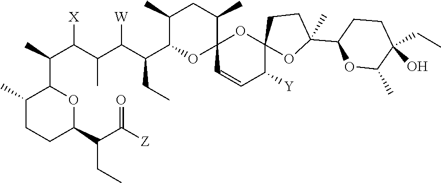
wherein X is OH, Z is OH, and Y is NR₁R₂ where R₁ is H and R₂ is selected from the group consisting of (C₁-C₁₆)-alkyl; (C₃-C₁₆)-alkenyl; (C₃-C₁₆)-alkynyl, and (C₃-C₁₆)-cycloalkyl.

22. The method of claim 21, wherein the iron chelator present in the pharmaceutical composition is a compound of formula (I): 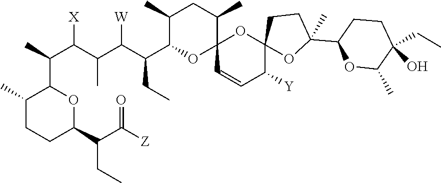
wherein W is ═O, X is OH, Z is OH, and Y is NR₁R₂ where R₁ is H and R₂ is selected from the group consisting of (C₃-C₅)-alkynyl and (C₃-C₆)-cycloalkyl.

23. A method for the treatment of a Mantle cell lymphoma (MCL) subject comprising simultaneous, separate, or staggered administration of a pharmaceutical combination product comprising:
(i) an iron chelator or a small molecule sequestering lysosomal iron; and
(ii) at least one other anti-cancer agent selected from the group consisting of agents used in chemotherapy, targeted treatments, immune therapies, and combinations thereof.

24. The method of claim 23, wherein the iron chelator or small molecule sequestering lysosomal iron is selected from the group consisting of Deferasirox, Deferoxamine, Deferiprone, Salinomycin, and analogs or derivatives thereof, and said other anti-cancer agent is selected from the group consisting of agents used in chemotherapy.

25. The method of claim 24, wherein the iron chelator is a nitrogen-containing analog of salinomycin of formula (I): 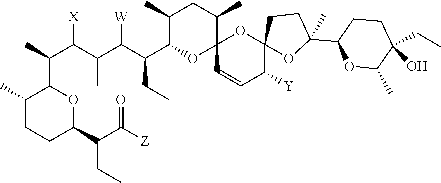
wherein W is ═O, X is OH, Z is OH, and Y is NR₁R₂ where R₁ is H and R₂ is selected from the group consisting of (C₃-C₅)-alkynyl and (C₃-C₆)-cycloalkyl, and the said chemotherapy compound is Doxorubicin, Venetoclax, or Ibrutinib.
